SEQUENCE LISTING (6 sequences)

SEQ ID NO: 1 - DNA, 20 bp, synthetic construct
  misc_feature 1..20: note = marker
  source 1..20: mol_type = other DNA
ggcagctgcgagagctcact

SEQ ID NO: 2 - DNA, 20 bp, synthetic construct
  misc_feature 1..20: note = marker
  source 1..20: mol_type = other DNA
ctgcgtggacaatggctact

SEQ ID NO: 3 - DNA, 402 bp, synthetic construct
  source 1..402: mol_type = other DNA
atggctcaggtgcagctggtggaaagcggcggctctctggtgcagcctggcggatctctgagactgagctgtgccgccagcggcagatttgccgagagcagcagcatgggctggttcagacaggcccctggcaaagaacgcgagttcgtggccgccatctcttggagcggcggagccaccaattacgccgatagcgccaagggccggttcaccctgagccgggacaacaccaagaacaccgtgtacctgcagatgaacagcctgaagcccgacgacaccgccgtgtactactgcgccgccaacctgggcaactacatcagcagcaaccagcggctgtacggctactggggccagggaacacaagtgaccgtgtccagccctttcacaggcggcggaggatcc

SEQ ID NO: 4 - DNA, 711 bp, synthetic construct
  source 1..711: mol_type = other DNA
agcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaa

SEQ ID NO: 5 - DNA, 2760 bp, Homo sapiens
  source 1..2760: mol_type = genomic DNA
gaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 6 - DNA, 1935 bp, Homo sapiens
  source 1..1935: mol_type = genomic DNA
atggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagaaaaattccgggttggcaattgcaagcatctcaaaatgaccagaccctga

(19) United States
(12) Patent Application Publication
(10) Pub. No.: US 2025/0231175 A1
(43) Pub. Date: Jul. 17, 2025

(54) LIVE CELL IMAGING PLATFORM FOR CONDENSATES

(71) Applicant: UMC UTRECHT HOLDING B.V., Utrecht (NL)

(72) Inventors: Boudewijn Marius Theodoor BURGERING, Utrecht (NL); Jurian SCHUIJERS, Utrecht (NL); Tianshu GUI, Utrecht (NL); Cassio Dejair FLEMING DE MORAES, Utrecht (NL)

(21) Appl. No.: 18/701,424

(22) PCT Filed: Oct. 20, 2022
(86) PCT No.: PCT/NL2022/050596

§ 371 Date: Apr. 15, 2024

(30) Foreign Application Priority Data
Oct. 20, 2021 (NL) 2029470

(51) Int. Cl. G01N 33/50; C07K 14/47; C07K 14/72; C07K 16/44; C12N 5/071; C12N 5/09

(52) U.S. Cl. CPC G01N 33/5011; C07K 14/47; C07K 14/721; C07K 16/44; C12N 5/0686; C12N 5/0693; C12N 5/0697; G01N 33/5082; C07K 2317/569; C07K 2319/60

(57) ABSTRACT

A method for observing biomolecular compensates by providing a cell expressing a first fusion protein comprising a protein that forms a component of the biomolecular condensate and a second fusion protein that comprises a nuclear localization signal is provided. The present invention further provides for methods and means for screening candidate agents by exposing cells expressing a first and second fusion protein with said candidate agents and observing the effects on biomolecular condensates.

### INTRODUCTION

Transcription regulation is an essential process during the development of organisms and in the maintenance of homeostasis in a cell. Transcription regulation allows for the cell to respond, for example by gene expression activation, modification or repression, to a variety of intracellular and/or extracellular signals or stimuli. Transcription regulation can occur as a result of a stimulus, for example a signaling protein, e.g. a transcription factor, that induces or activates a particular intracellular signal transduction pathway. Intracellular signal transduction pathways commonly start off as a response to such an intracellular and/or extracellular signal, e.g. the binding of a signaling molecule to a transmembrane receptor protein, in a single or a cascade of simultaneous or sequential biochemical changes in cellular molecules ultimately resulting in a culmination of signaling pathways in the cell nucleus. Once in the nucleus, a key signaling protein can modulate the activity of target genes.

Dysregulation of intracellular signaling pathways is a common driving event in a variety of diseases and health conditions, including oncogenesis. Such dysregulation of signaling pathways can result in the abnormal activation or modulation of target oncogenes or abnormal repression or modulation of tumor suppressor genes. Ultimately, abnormal signaling can result in abnormal cellular behavior, abnormal growth and eventually cancer.

Research over the past decades has revealed fundamental roles in biochemical and cellular processes, such as in intracellular signaling pathways, for membraneless intracellular assemblies of multivalent molecules commonly referred to as biomolecular condensates (Banani, S. et al., Nat Rev Mol Cell Biol 18, 285-298 (2017), Hnisz D et al. Cell Volume 169(1), 13-23 (2017), Shin Y, Brangwynne C P, Science (2017), Alberti S. et al., Nat Rev Mol Cell Biol 22, 196-213 (2021), Boija A et al., Cancer Cell 39(2), 174-192 (2021), Sabari B R, Dev Cell 55(1), 84-96 (2020)). The major research focus (see also Schuster B et al., J. Phys. Chem. B 2021, 125, 3441-3451) has been the identification of the molecular features that lead to condensate formation, including the study of affinity features (Martin et al., Emerg Top Life Sci. 4(3), 307-329 (2020), Wang et al., Cell. 2018 174(3), 688-699 (2018)), charge distribution (Sherry et al., Proc. of the Nat. Acad. of Sci. 114(44), E9243-E9252 (2017)). However, understanding about the cellular mechanisms that regulate, modulate, stimulate or inhibit biomolecular condensate formation, and/or that regulate, modulate, stimulate or inhibit maintenance or dissolution of these condensates is still in its infancy.

One of the underlying reasons as to why so little is still known about formation, maintenance, perturbation and dissolution of these biomolecular condensates is the lack of reliable detection methods. Present methods for observing biomolecular condensates included methods that rely on the detection of signaling pathways and changes in gene expression. Methods presently used to identify biomolecular condensates lack in reliability because these methods, for example, rely on transcriptional regulation driven by intracellular signaling pathways to activate reporter genes such as luciferase or fluorescent proteins. Consequently, presently used detection methods suffer from some serious issues, namely 1) the methods are indirect, because they can also be activated through secondary effects e.g. effects affecting activation, transcription etc. of the genes, 2) the methods are unresponsive because reporters are retained after the removal of the intracellular signaling, 3) the methods do not detect biomolecular condensates (e.g. formed as the consequence of condensation of different biomolecules) and/or 4) the methods are slow due to the multi-step process before any signal even is detectable. Consequently, there is a need for an improved, fast and reliable detection methods for biomolecular condensates.

### SUMMARY OF THE INVENTION

The current inventors now provide for a new, reliable, easy-to-perform and inventive method for observing biomolecular condensates. Most methods available in the art for detecting biomolecular condensates, although useful, are rather restricted in that they rely on the activation of fluorescent markers by means of transcription driven by cell signaling pathways, which makes such methods largely unsuitable for identifying biomolecular condensates, even more so for live detection of said condensates. In addition, there are methods available in the art that relate to study protein-protein interaction and rely on providing the cell with artificial constructs in order to provide and/or over-express the binding partner(s) for which the interaction is to be determined or studied. In contrast, the current invention allows for the detection of naturally occurring, endogenous biomolecular condensates, including nascent or pre-assembled biomolecular condensates, including biomolecular condensates present in the cell before the first and second fusion proteins of the invention are provided to the cell. In other words, the current invention is aimed at detecting endogenous and/or intracellular and/or natural expression and/or formation of such, preferably naturally occurring biomolecular condensates, and changes therein. Furthermore, the inventors surprisingly found that improving the method for detecting biomolecular condensates according to the present disclosure enables the detection/observation of the formation, presence, maintenance, perturbation and/or dissolution of biomolecular condensates in live cells. The method of the present invention is highly responsive to changes in stimuli that modulate biomolecular condensate formation, maintenance, perturbation and/or dissolution. For example, it was found that with the method of the present invention it is now for the first time possible to immediately detect changes, such as formation, maintenance, perturbation and/or dissolution of different types of biomolecular condensates in response to a stimulus that leads to the activation of a signaling pathway, e.g. a signaling pathway that leads to the formation of biomolecular condensates, for example the Wnt-signaling pathway. At the same time it was found that with the method of the present invention it is now also for the first time possible to immediately detect such changes when the stimulus that leads to the activation of a signaling pathway is removed, and/or when an antagonist (or inhibitor) of the signaling pathway, leading to (partial) deactivation of the signaling pathway, is used.

Overall, the inventors found that with the method of the present invention, the detection of biomolecular condensates (including its formation, maintenance, perturbation and/or dissolution) is highly improved by allowing to detect immediate changes in response to modulation stimuli. With the method of the present invention, it has therefore now become possible to also screen for compounds that have a direct effect on biomolecular condensate formation, maintenance, perturbation and/or dissolution. Not only does this allow to identify candidate agents for pharmaceutical use (e.g. in cancers involving disturbed, mutated, dysregulated signaling pathways,) but also allow for a better understanding of the mechanism-of-action of (pharmaceutical) agents in relation to biomolecular condensates.

Hence in one embodiment of the present invention, a method of observing biomolecular condensates is provided, wherein the method comprises:
(a) providing at least one cell wherein the cell expresses
   (i) a first fusion protein, wherein the first fusion protein comprises a first part and a second part, wherein the first part and the second part are linked together, wherein the first part comprises a protein that forms a component of the biomolecular condensate, wherein the second part comprises a protein capable of binding to a cognate binding partner, and
   (ii) a second fusion protein, wherein the second fusion protein comprises a first part and a second part, wherein the first part and the second part are linked together, wherein the first part and/or the second part comprises the cognate binding partner, wherein the first part comprises a localization signal, wherein the second part comprises a fluorescent protein; and
(b) observing the presence of the biomolecular condensates by detecting the fluorescent protein comprised in the second part of the second fusion protein.

By the method of the invention biomolecular condensates can be observed and detected in real-time. It was found that the method of the invention is highly responsive in detecting biomolecular condensates, including detecting changes in the biomolecular condensates, including formation, maintenance, perturbation and/or dissolution. The present inventors also found that with the method of the invention it is no longer needed to rely on the production and/or degradation of reporter molecules in activating or deactivating signaling pathways involving biomolecular condensates. The method of the invention is therefore highly useful for observing the formation, maintenance, perturbation and/or dissolution of said biomolecular condensates and thus improving the understanding of these biomolecular condensates. In the method of the invention, participation of the first fusion protein in a biomolecular condensate can be observed. The first fusion protein comprises a first part that comprises a protein known or expected to be a component of a biomolecular condensate (i.e. known or expected to form part of the biomolecular condensate of interest). Participation of the first fusion protein in the biomolecular condensate is detected by binding of the second fusion protein, comprising a fluorescent protein, to the first fusion protein. As can be witnessed from the examples, the biomolecular condensates can be observed as distinct foci in cells using fluorescent microscopy.

The method of the present invention is in particular suitable for observing biomolecular condensates under conditions wherein the protein that is comprised in the first part of the first fusion protein is a protein that becomes part of a biomolecular condensate in response to a stimulus, for example, a stimulus leading to the activation of a signaling pathway. For example, such protein may normally mainly reside outside a biomolecular condensate, but upon providing the stimulus translocates within the cell, for example towards the nucleus, and participates in the formation of a biomolecular condensate. With the method of the present invention such translocation of a protein (as being comprised in the first part of the first fusion protein) can be readily detected using e.g. fluorescent microscopy, as the consequence of the presence of the second fusion protein that can bind with the first fusion protein. In other words, the method of the current inventions allows to capture the translocation of a protein from one cellular compartment to another and its participation in a biomolecular condensate. It was advantageously found that by providing the second fusion protein with a localization signal, e.g. a nuclear localization signal, and wherein said localization corresponds to the cellular localization (e.g. in the nucleus) wherein the biomolecular condensate of interest is formed or resides, detection of the presence, formation, maintenance, perturbation and/or dissolution of the biomolecular condensate is highly improved and allows for real-time detection in changes in such biomolecular condensates. For example, in some preferred embodiments, the protein that is comprised in the first part of the first fusion protein is normally mainly present in the plasma membrane of a cell, and does mainly not participate in a biomolecular condensate in the absence of a stimulus activating a particular signaling pathway. Upon providing the stimulus, the protein that is comprised in the first part of the first fusion protein translocates into the nucleus and becomes a component of a biomolecular condensate, which biomolecular condensate than becomes visible by binding of the second fusion protein to the first fusion protein, which second fusion protein mainly resides in the nucleus as the consequence of the presence of its localization signal (in this case nuclear localization signal).

Therefore, the method of the present invention is uniquely capable of observing live translocation to the nucleus and participation in biomolecular condensates. Therefore, in a preferred embodiment the method comprises that the first part of the first fusion protein is a protein that translocates from one location in a cell to a second location, preferably into a cell nucleus in response to a stimulus, preferably wherein the translocation is from the plasma membrane into the cell nucleus and/or wherein the translocation is from the cytosol into the cell nucleus. As a consequence of the stimulus the first fusion protein will, for example, translocate from the plasma membrane to the nucleus.

The inventors also found that the method of the invention is particularly beneficial in the identification of compounds and/or agent that may interfere with the biomolecular condensates, for example that modulate (e.g. inhibit or stimulate) formation, maintenance, perturbation and/or dissolution of said biomolecular condensates.

Hence, in another embodiment of the invention a screening method for identifying a compound that modulates the formation, maintenance and/or dissolution of a biomolecular condensate is provided, wherein the method comprises:
(a) providing at least one cell wherein the cell expresses;
   (i) a first fusion protein, wherein the first fusion protein comprises a first part and a second part, wherein the first part and the second part are linked together, wherein the first part comprises a protein that forms a component of the biomolecular condensate, wherein the second part comprises a protein capable of binding to a cognate binding partner, and
   (ii) a second fusion protein, wherein the second fusion protein comprises a first part and a second part, wherein the first part and the second part are linked together, wherein the first part and/or the second part comprises the cognate binding partner, wherein the first part comprises a localization signal, wherein the second part comprises a fluorescent protein;
(b) contacting the cell with a candidate agent; and
(c) detecting a change in the formation, maintenance, and/or dissolution of the biomolecular condensate compared to a control by detecting the fluorescent protein comprised in the second part of the second fusion protein to determine the candidate agent's modulatory activity.

It is understood herein that said method preferably is to identify signaling pathway modulators, such as, but not particularly limited to, Wnt signaling pathway modulators, JAK/STAT signaling pathway modulators, RAS signaling pathway modulators, HIPPO signaling pathway modulators, TGF-β signaling pathway modulators, or nuclear receptor signaling modulators. Since in the method of the invention a cell is modified as to express a novel first and second fusion protein, the invention also provides for at least one cell wherein the cell expresses:
(a) a first fusion protein, wherein the first fusion protein comprises a first part and a second part, wherein the first part and the second part are linked together, wherein the first part comprises a protein that forms a component of the biomolecular condensate, wherein the second part comprises a protein capable of binding to a cognate binding partner,
(b) a second fusion protein, wherein the second fusion protein comprises a first part and a second part, wherein the first part and the second part are linked together, wherein the first part and/or the second part comprises the cognate binding partner, wherein the first part comprises a localization signal, wherein the second part comprises a fluorescent protein.

### BRIEF DESCRIPTION OF THE DRAWINGS

FIG. 1:

A) Left: Schematic illustration of the EGFP-nanobody-B-catenin imaging system. NLS-EGFP is expressed under a constitutive promoter and accumulates in the nucleus. beta-catenin is fused with a nanobody targeting EGFP. Upon nuclear translocation EGFP is recruited by the nanobody-beta-catenin fusion protein. Clustering of EGFP: nanobody-beta-catenin complexes allows detection of beta-catenin condensates. Right: Schematic illustration of the experimental setup. Anti-EGFP nanobody was fused to beta-catenin in the endogenous locus of HEK293T cells. NLS-GFP was expressed using the endogenous H2BC17 allele, using a T2A sequence to separate the gene products.

B) Time-lapse images of GNB cells after stimulation with 3 μM CHIR. Scale bar, 5 μm. Mean of the number of foci per cell nucleus after addition of 3 μM CHIR. Black line represents the mean of at least 100 nuclei. Grey area indicates the standard error of the mean (SEM).

C) Time-lapse images of GNB cells after media change omitting CHIR. Scale bar, 5 μm.

FIG. 2: shows results obtained with an agent that was identified to promote dissolution of β-catenin condensates, as witnessed by the reduced number of foci per nucleus in the presence of the compound (right) in comparison to control (left). Treatment of cells with said component lead to dissolution as can be observed using the method described herein. These results show that with the method of the invention candidate agents can be identified that modulate formation, maintenance and/or dissolution of biomolecular condensates, as exemplified by the dissolution of the B-catenin condensates in the presence of the compound.

FIG. 3: GNB system detects b-catenin condensates (green, EGFP, top row dashed arrows) that are distinct from several nuclear bodies and are sites of active transcription

A) Immunofluorescence images of well-known nuclear bodies (red, mCherry, second row from top, continuous arrow) on GNB cells (green) after 24 hours of 3 μM CHIR stimulation. Scale bar, 5 μm. The line plots at the bottom show the IF intensities of the B-catenin foci and the indicated nuclear condensates along the lines indicated in in the merged panel.

B) Images of labeling of nascent RNA (red, most left panel) after a 10 minute EU (5-Ethynyl Uridine) (pulse in the GNB cells. B-catenin condensates (in green, second panel) co-localize with the largest spots of RNA production.

C) Panel shows quantification of nascent RNA signal across 15 b-catenin condensates. Nascent RNA specifically co-localizes with b-catenin condensates. Scale bar 5 μm.

FIG. 4: GNB system detects b-catenin condensates that behave like liquid droplets A) Representative images of the FRAP analysis in GNB-B-catenin droplets 24 hours after 3 μM CHIR stimulation. Scale bar, 1 μm. FRAP was performed with a confocal microscope using a 100× magnification.

B) Line graph shows relative mean intensity change before and after photobleaching. Data plotted are mean±SEM, n=6.

C) Representative images of live imaging of GNB-β-catenin cells showing droplets fusion (arrowhead) at indicated time points (minutes). Scale bar, 5 μm.

D) Co-staining for active RNA Polymerase II (poIII) and for the DNA-resident b-catenin recruiting factors TCF7 and TCF7L2. Active poIII is present in distinct condensates throughout the nucleus and all b-catenin condensates overlap with active poIII condensates, and one of the antibodies showed a specific enrichment over random control (FIG. 4D). Both TCF7 and TCF7L2 stainings also showed a high number of nuclear condensates and all b-catenin condensates overlap with at least one TCF condensate (FIG. 4D).

FIG. 5: GFP-nanobody-βcatenin system on organoids. Tumor progression organoids (TPO) showing the formation of condensates (arrowheads) after WNT-stimulus. The figure shows the GNb system described herein but now in primary organoid cultures. This expands the system to incorporate patient derived material.

FIG. 6: mCherry-nanobody system for the Androgen Receptor pathway. a) Schematic representation of the experiment. HEK293T cells were co-transfected with mCherry together with either mCherry-nanobody-AR or the splicing variant ARV7, which is constitutively active. After one day, cells were treated with different concentrations of Dihydrotestosterone (DHT). b) Representative images of mCherry condensates seen in the cells. This experiment shows the invention is broadly applicable to different types of condensates, including disease relevant condensate. The experiment also shows the invention is broadly applicable using different types of fluorescent proteins or fluorophores.

### DESCRIPTION

The section headings used herein are for organizational purposes only and are not to be construed as limiting the subject matter described.

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein. For purposes of the present invention, the following terms are defined below.

As used herein, the singular forms “a,” “an” and “the” include plural referents unless the context clearly dictates otherwise. For example, a method for administrating a pharmaceutical agent includes the administrating of a plurality of molecules (e.g. 10's, 100's, 1000's, 10's of thousands, 100's of thousands, millions, or more molecules).

The terms “about” and “approximately”, when referring to a measurable value such as an amount, a temporal duration, and the like, is meant to encompass variations of ±20% or ±10%, more preferably ±5%, even more preferably ±1% and still more preferably ±0.1% from the specified value, as such variations are appropriate to perform the disclosed methods.

As used herein, the term “and/or” indicates that one or more of the stated cases may occur, alone or in combination with at least one of the stated cases, up to with all of the stated cases.

As used herein the term “binding partner”, for example as used in the term “cognate binding partner”, refers to a (part of a) (macro) molecule, such as a protein or nucleic acid that is a part of a binding pair. A binding pair comprises two binding partners that bind to each other through for example, but not limited to, electrostatic bonding, hydrophobic bonding, van der Waals forces, steric interactions, salt bridges etc. Well-known examples of binding partners, e.g. cognate binding partners, are a receptor and a cognate ligand, or an antibody and a cognate protein (antigen).

As used herein the term “biomolecular condensate” are known by the skilled person and refers to a membraneless intracellular assembly of multivalent molecules, e.g. proteins, nucleic acids etc., normally in the nucleoplasm and/or cytoplasm that can be formed by liquid-liquid phase separation. Such membraneless intracellular assembly is in the art sometimes also referred to phase-separated biomolecular condensates. Preferably the biomolecular condensate is a biomolecular condensate in the nucleoplasm (nucleus).

In general, size of the condensate (including the biomolecular condensates of the invention) is referred to as ‘meso scale” which refers to anything that is larger than nanometer scale (molecular protein-protein interactions), but smaller than millimeter scale (whole cells and cell-cell interactions). In some embodiments, the condensates detected with the method of the invention, for example using the GNb system described herein, are in the high nanometer, low micrometer scale, e.g. they are smaller than nucleoli (see also Banani, S. F., Lee, H. O., Hyman, A. A., and Rosen, M. K. (2017). Biomolecular condensates: organizers of cellular biochemistry. Nat. Rev. Mol. Cell Biol. 18, 285-298. 10.1038/nrm.2017.7.; Sabari, B. R., Dall'Agnese, A., and Young, R. A. (2020). Biomolecular condensates in the nucleus. Trends Biochem. Sci. 45, 961-977. 10.1016/j.tibs.2020.06.007.). In some embodiments, the biomolecular condensates detected according to the invention may have a size of at least (with increasing preference) 5, 10 or 20 nm, preferably at least 30 nm, for example at least 50, 60, 70, 80 or 100 nm, or larger. In some embodiment the condensates detected according to the invention may have a size smaller than 1500 nm, 1000 nm, 500 nm, or 250 nm, including smaller than 150 nm, 100 nm, or 80 nm.

As used the term “cancer” refers to a malignant diseased state of a subject that is characterized by an aberrant growth and/or aberrant division of a cell in a tissue. Cancerous tissue (neoplasm) can comprise cells that can spread and invade other tissues of the subject's body (metastasize). When referring herein to a “cancer cell” a cell exhibiting an aberrant growth pattern and/or aberrant cell division is meant.

As used herein the term “component” refers to a part or an element of a larger whole, e.g. a constituent. In relation to biomolecular condensates a component is meant to refer to distinct molecules, e.g. proteins, nucleotides or other molecules, that can contribute, for example through interactions, to the formation and stabilization of a biomolecular condensate.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein the term “fusion protein” refers to a protein comprising at least two protein domains (e.g. parts). Said protein domains can be encoded by separate genes that have so been joined, for example by genetic engineering, as to be transcribed and translated as a single unit, producing a single polypeptide. The separate parts of the fusion domain may be adjacent to each other or may be separated by further domain, for example by a linker protein or sequence, linking the first domain and the second domain together.

As used herein the term “modulation”, and also the related verb “modulate” or variations thereof such as “modulates” or “modulating”, such as in the context of “a modulating compound” will be understood as referring to the process of affecting, altering or changing, for example as a result of an interaction with “a modulating compound”, of function, status or structural configuration of something, such as one or more molecules, proteins or, within the context of the current invention, biomolecular condensates. For example, within the context of the current invention, a compound may modulate, i.e. promote, enhance, reduce, inhibit, slow-down formation, maintenance and/or dissolution of a biomolecular condensate.

As used herein the term “translocation”, and also the related verb “translocate” or variations thereof such as “translocates” or “translocating”, for example such as in the context of “protein translocation” will be understood as meaning the process by which molecules, e.g. proteins, move between cellular compartments, e.g. cell membrane, cytosol, nucleus etc. For example, within the context of the current invention, translocation may refer to the movement of a protein between cellular compartments (e.g. the plasma membrane and the nucleus) in response to a compound that activates (stimulus) a signaling pathway causing the movement of said protein.

### DETAILED DESCRIPTION

In a first embodiment, a method is provided for observing biomolecular condensates, wherein the method comprises:
(a) providing at least one cell wherein the cell expresses:
(i) a first fusion protein, wherein the first fusion protein comprises a first part and a second part, wherein the first part and the second part are linked together, wherein the first part comprises a protein that forms a component of the biomolecular condensate, wherein the second part comprises a protein capable of binding to a cognate binding partner, and
(ii) a second fusion protein, wherein the second fusion protein comprises a first part and a second part, wherein the first part and the second part are linked together, wherein the first part and/or the second part comprises the cognate binding partner, wherein the first part comprises a localization signal, wherein the second part comprises a fluorescent protein; and
(b) observing the presence of the biomolecular condensates by detecting the fluorescent protein comprised in the second part of the second fusion protein.

As is known to the skilled person, biomolecular condensates are assemblies of molecules, e.g. proteins, nucleic acids etc., in the nucleoplasm and/or cytoplasm of a cell. Biomolecular condensates are often formed by liquid-liquid phase separation. Molecular interactions that lead to biomolecular condensate formation have been shown to be complex and include combinations of electrostatic, cation-p, p-p, hydrogen bonding and hydrophobic interactions (Boija et al. Cell (2018) 175, 1842-1855.e16, Das et al. Curr. Opin. Struct. Biol. (2015) 32, 102-112. Martin et al. Science (2020) 367, 694-699. Nott et al. Mol. Cell (2015) 57, 936-947, Pak, et al. Mol. Cell (2016) 63, 72-85. Vernon et al. Elife (2018) 7, Wang, et al. Cell (2018) 174, 688-699.e16). A further review on the skilled persons understanding of biomolecular condensates is provided by Borcherds et al. Curr Opin Struct Biol. 2021 April; 67:41-50. Biomolecular condensates regularly occur as a result of culmination of signaling pathway-related molecules such as, but not limited to, transcription factors.

Such culmination is likely to occur at a location in the nucleus where the key signaling molecule can induce activation or repression of one or more target genes.

Within the context of the current invention, the biomolecular condensate may reside in any suitable cellular compartment, including cytoplasm, e.g. cytoplasmic condensates. However, it is preferred the biomolecular condensate is a nuclear biomolecular condensate, i.e. a condensate that is, for example, formed in the nucleus, for example in response to modulation of a signaling pathway, such as those disclosed herein, for example, in response to a stimulus of the signaling pathway. Therefore, in a preferred embodiment, a method is provided for observing nuclear biomolecular condensates, wherein the method comprises:
(a) providing at least one cell wherein the cell expresses:
(i) a first fusion protein, wherein the first fusion protein comprises a first part and a second part, wherein the first part and the second part are linked together, wherein the first part comprises a protein that forms a component of the nuclear biomolecular condensate, wherein the second part comprises a protein capable of binding to a cognate binding partner, and
(ii) a second fusion protein, wherein the second fusion protein comprises a first part and a second part, wherein the first part and the second part are linked together, wherein the first part and/or the second part comprises the cognate binding partner, wherein the first part comprises a nuclear localization signal, wherein the second part comprises a fluorescent protein; and
(b) observing the presence of the nuclear biomolecular condensates by detecting the fluorescent protein comprised in the second part of the second fusion protein.

In some embodiments, multiple first fusion proteins and multiple second fusion proteins are present in at least one cell in the method in accordance with the invention. In other words, multiple copies of a first fusion protein and multiple copies of a second fusion protein may be present in one cell and/or in multiple cells within the scope of the method of the invention. In some embodiment, more than one type of first fusion protein and/or more than one type of second fusion protein may be utilized, for example in order to simultaneously detect the presence of one or more different types of biomolecular condensates.

It is understood that said two fusion proteins can be expressed by a cell by, for example, modifying the genome of said cells so that the fusion proteins are expressed by said cell. Methods for designing and expressing fusion proteins are well-known in the field and the skilled person is well able to apply conventional techniques for allowing the cell to express the fusion protein of the present invention. Expression may be transient but also be continuous of in response to an expression signal./pct

It is understood herein that the first fusion protein and/or the second fusion protein comprises a first and a second part. In some embodiments it is preferred that at least one of said part of a first fusion protein and/or at least one part of a second fusion protein is a protein (or part thereof) that is native to the cell in which it is expressed. For example, the first part of the first fusion protein comprises a protein that forms a component of the (nuclear) biomolecular condensate. Although is some embodiments such protein may not be native to the cell in which the fusion protein is expressed, in a preferred embodiment, the protein that is comprised in the first part of the first fusion protein is a protein that naturally occurs in the cell (e.g. is of the same species). The same applies in particular for the (nuclear) localization in the first part of the second fusion protein.

The first fusion protein and a second fusion protein both comprise a first part and a second part. These first and second parts in a fusion protein are preferably linked or connected together. A skilled person is aware of methods to connect or link two separate domains or parts of a fusion protein and is skilled in the use of such methods. Such methods comprise for example, but not limited to, the use of small, non-polar or polar linker amino acids, large structural alpha-helix forming linkers etc.

The first fusion protein comprises a first part and a second part. The first part of the first fusion protein comprises at least one protein, wherein said protein forms a component of a biomolecular condensate. In other words, the first part of said first fusion protein comprises at least one protein, or part thereof, that is part of a biomolecular condensate (i.e. that can become part of a biomolecular condensate upon formation of such biomolecular condensate)./pct

It is understood herein that in preferred embodiments of the invention, the protein that is comprised in the first part of the first fusion protein may become part of a biomolecular condensate (i.e. is a component of the biomolecular condensate), for example in response to a stimulus activating a signaling pathway. Accordingly, it is understood that the first fusion protein, may means of the protein comprised in the first part of the first fusion protein, may likewise become part of the biomolecular condensate, for example, in response to the activation of a signaling pathway, and wherein said signaling pathway normally causes the protein that is comprised in the first part of the first fusion protein to participate in the formation (or, e.g. growth) of the biomolecular condensate. In other words, the first fusion protein that comprises such protein that can be a component of a biomolecular condensate will also localize, for example in response to a stimulus, to a biomolecular condensate, for example in the cytoplasm and/or nucleoplasm of the cell.

In a preferred embodiment of the invention, the first part of the first fusion protein forms a component of a nuclear biomolecular condensate.

The second part of the first fusion protein comprises at least one protein that is capable of binding to a cognate binding partner. As meant herein “binding” refers to the forming an interaction between one molecule, for example a protein, and a complementary molecule, for example a cognate binding protein. Said second part of the first fusion protein is linked or connected to the first part of the first fusion protein. It is contemplated that the second part of the first fusion protein will, together with said first part, also localizes to a biomolecular condensate and/or become a part of the said biomolecular condensate once the protein comprised in the first part of the fusion protein translocates to said biomolecular condensate.

The second fusion protein comprises a first part and a second part, wherein the first part and the second part are linked together, wherein the first part and/or the second part comprises the cognate binding partner to which the second part of the first fusion protein may bind. In some embodiments, the first part of the second fusion protein comprises at least one protein, wherein said first part may comprise the cognate binding partner of the second part of the first fusion protein and wherein said first part is a nuclear localization signal. Similarly, in some embodiments, the second part of the second fusion protein comprises at least one protein, wherein said second part may comprises the cognate binding partner and wherein said second part comprises a fluorescent protein.

It is understood herein that either the first part and/or the second part of the second fusion protein may comprise the cognate binding partner of the second part of the first fusion protein. As such, the protein that comprises the second part of said first fusion protein may bind with a cognate binding partner selected from the first part and/or the second part of the second fusion protein. Preferably, the cognate binding partner is comprised in the second part of the second fusion protein, i.e. wherein the fluorescent protein that is comprised in the second part of the second fusion protein is the cognate binding partner. When the protein comprising the second part of the first fusion protein and its cognate binding partner bind to each other, the first fusion protein can be detected based on the detection of the fluorescent protein. When the first fusion protein is part of a biomolecular condensate, the biomolecular condensate is likewise detected, and may, for example, be observed as well-recognizable foci, for example, using fluorescence microscopy. As such, in the method of the invention there is provided for observing the presence of the biomolecular condensates by detecting the fluorescent protein comprised in the second part of the second fusion protein.

With the method of the invention it has thus now become possible to visualize the formation, maintenance, or dissolution of biomolecular condensates, and thereby also visualize directly the activity of signaling pathway that leads to, for example, the formation of biomolecular condensates, for example, in response to activation or modulation of signaling cascades/pathways, including those described herein.

It is contemplated that biomolecular condensates can be observed and detected in the cell as a whole. By using the method of the invention it is now possible to directly detect biomolecular condensates in the nucleus (e.g., but not limited to, nucleolus, nuclear speckles, PML bodies, Histone Locus bodies, RNA-Polymerase II condensates and Cajal bodies) and/or in the cytosol (e.g., but not limited to germ granules, processing bodies (P-bodies), stress granules (SGs), Lewy bodies, glycogen granules, signalosomes and RNP transport granules) and that these biomolecular condensates can be identified and monitored, for example in relation to the modulation of one or more signaling pathways in the cell, for example by providing a stimulus that modulates such signaling pathway. Biomolecular condensates in the nucleus can comprise proteins that have translocated from the cytosol into the nucleus of a cell, for example as a response to an intracellular and/or extracellular stimulus.

Further examples of the biomolecular condensates that can be observed using the method of the invention are known to the skilled person and include those described by, for example, Banani, S. F., Lee, H. O., Hyman, A. A., and Rosen, M. K. (2017). Biomolecular condensates: organizers of cellular biochemistry. Nat. Rev. Mol. Cell Biol. 18, 285-298. 10.1038/nrm.2017.7; Sabari, B. R., Dall'Agnese, A., and Young, R. A. (2020). Biomolecular condensates in the nucleus. Trends Biochem. Sci. 45, 961-977. 10.1016/j.tibs.2020.06.007; Boija, A., Klein, I. A., and Young, R. A. (2021). Biomolecular condensates and cancer. Cancer Cell 39, 174-192. 10.1016/j.ccell.2020.12.003., including Synaptic densities, RNA transport granule, Balibani body, Stress granule, P-body, U-body, Germ granule, Polycomb Body, Nuclear Gem, OPT domain, paraspeckle, CTCF clusters, DNA damage foci, PML body, Cajal body, Paraspecle, Cleavage Body, and Nuclear pore complex

Therefore, in a preferred embodiment a method in accordance with the invention is provided, wherein the method comprises that the protein comprised in the first part of the first fusion protein is a protein that translocates into a cell nucleus in response to a stimulus, preferably wherein the translocation is from the plasma membrane into the cell nucleus and/or wherein the translocation is from the cytosol into the cell nucleus.

Cellular signaling is a fundamental property of a cell in its ability to respond to intracellular and/or extracellular events that directly and/or indirectly influence the homeostasis of a cell. Examples of extracellular signals are physical agents, such as mechanical pressure, voltage, temperature, light, or chemical signals (e.g., small molecules, peptides, or gas), but also comprise endocrine signals, such as hormones, or agonists and/or antagonists of intracellular or cell surface receptors involved in cellular signaling.

In preferred embodiments of the method of the invention the protein comprised in the first part of the first fusion protein is a protein that, for example, in its native form, responds to a stimulus by translocation from, for example, the plasma membrane, to, for example, the nucleus and where it can become part of a biomolecular condensate that is formed, for example, in the nucleus, in response to said stimulus. Such stimulus can be any signal or agent provided to the cells and that directly and/or indirectly influences a cell, e.g. by affecting a cell's homeostasis, and has the effect that it triggers translocation of a protein comprised in the first part of the first fusion protein according to the invention. Translocation of the protein comprised in the first part of the first fusion protein can be to any other location in the cell. For example, said protein may translocate in response to a stimulus from one location in the cytosol to another location in the cytosol. In another example, said protein can, in response to a stimulus, for example by a receptor-induced signal, translocate from the plasma membrane of the cell to the cytosol or to the nucleus of said cell. As such it is one preferred aspect method in accordance to the invention that the method comprises that the protein comprised in the first part of the first fusion protein is a protein that translocates from the plasma membrane into the cell nucleus in response to a stimulus. It is another preferred aspect of the method in accordance to the invention that the method comprises that the protein comprised in the first part of the first fusion protein is a protein that translocates from the cytosol into the cell nucleus in response to a stimulus. It can also be that the first part of the first fusion protein is a protein that translocates in a first stage of the translocation from the plasma membrane into the cytosol and in a further stage, e.g. in response to a further stimulus or in response to continued stimuli, from the cytosol to the nucleus of the cell. In a preferred aspect of the invention said proteins are such proteins that are, or are associated with, plasma membrane proteins, preferably signal transduction proteins. The stimulus may, for example lead to the translocation of the protein as the consequence of modulation, e.g. activating or inhibiting, a particular signaling pathway in the cell.

Therefore, in a further embodiment a method in accordance with the invention is provided, wherein the protein comprised in the first part of the first fusion protein is a protein selected from the group consisting of a protein of the Wnt signaling pathway, JAK/STAT signaling pathway, RAS signaling pathway, the HIPPO signaling pathway, TGF-β pathway or the Nuclear hormone receptor family.

The Wnt signaling pathway comprises a group of signal transduction pathways that are activated by the binding of a Wnt-protein ligand to a part of a receptor, i.e. the N-terminal extra-cellular cysteine-rich domain of a Frizzled family receptor, which passes the stimulus triggered by the binding of said ligand to the receptor domain, to a cytoplasmic protein, i.e. Disheveled (Dsh) protein (Komiya Y, Habas R. Wnt signal transduction pathways. Organogenesis. 2008; 4(2):68-75.). It is preferred that the protein comprised in the first part of the first fusion protein is a protein which is, or is associated with, plasma membrane proteins. It is highly preferred in the method of the invention that said protein is a protein associated with the canonical Wnt pathway. The canonical Wnt pathway (or Wnt/β-catenin pathway) is the Wnt signaling pathway that causes an accumulation of β-catenin in the cytosol and/or nucleus and its eventual translocation into the nucleus to act as a transcriptional coactivator of transcription factors that belong to the TCF/LEF family (Buechling & Boutros, Current Topics in Developmental Biology, Academic Press, Volume 97, Pages 21-53 (2011))

The JAK/STAT signaling pathways comprises Janus kinases (JAKs), and signal transducer and activator of transcription proteins (STATs). Upon a stimulus, comprising the binding of a ligand to the receptor, JAKs phosphatizes the receptor, allowing the binding of STAT proteins. Consequently, the STAT proteins are phosphorylated by JAKs and form a dimer. The dimer of STATs can translocate to the cytosol and/or nucleus (Cha & Schindler, Encyclopedia of Biological Chemistry, Elsevier, Pages 491-496 (2004)). STAT proteins can be components of a biomolecular condensate, preferably of a nuclear biomolecular condensate. Said biomolecular condensate can bind DNA and activate transcription of target genes of the JAK/STAT signaling pathway.

The RAS signaling pathway comprises the MAPK/ERK pathway, which can be defined by a RAF-MEK-ERK signaling axis. The pathway can be activated by a number of receptors including, but not limited to, receptor tyrosine kinases (RTKs), G-protein coupled receptors (GPCRs), and integrin family members. RAS activation is triggered by the assembly of scaffolding proteins. RAS family members display distinct post-translational modifications, which regulate their subcellular translocation, e.g. translocation to the plasma membrane (Gimple R C and Wang X, Front. Oncol. 9:965 (2019)).

The HIPPO signaling pathway is a pathway involved in cell proliferation and apoptosis. The pathway comprises a core kinase cascade wherein Mst1/2 (mammalian ortholog of Drosophila Hippo) kinases and SAV1, upon receiving a stimulus for example by upstream molecules such as Merlin, KIBRA, RASSFs, and Ajuba, form a complex to phosphorylate and activate LATS1/2 (mammalian ortholog of Drosophila Warts (Wts) LATS1/2 kinases in turn phosphorylate YAP and/or TAZ. The transcription proteins YAP and/or TAZ translocate into the nucleus and interact with other transcription factors to induce expression of target genes, in particular target genes involved in promoting cell proliferation and inhibition of apoptosis (Badouel C, McNeill, SnapShot: The hippo signaling pathway. Cell 145(3). 484-484.e1 (2011))

The TGF-β pathway comprises a pathway involved in numerous cellular processes, including but not limited to, cell differentiation, cell growth, apoptosis, cell migration, homeostasis. In said TGF-β signaling pathway the cellular processes are regulated by a signaling transduction pathway comprising the binding of ligands, e.g. TGF-β superfamily ligands, to a type II receptor. Said binding triggers the recruiting and phosphorylation of a type I receptor, which in turn phosphorylates SMADs (e.g. SMAD1, SMAD2, SMAD3, SMAD5, SMAD8), that can, in turn, bind coSMAD. R-SMAD/coSMAD complexes translocate and accumulate in the nucleus. Here these complexes act as transcription factors and participate in the regulation of target gene expression (Massagué, J. Nat Rev Mol Cell Biol 13, 616-630 (2012)).

The nuclear hormone receptor family comprises a family of proteins that can respond to molecules, comprising hormones, or hormone-analogues, such as for example, but not limited to, steroid hormones or thyroid hormones. Examples of nuclear hormone receptors are Androgen Receptor (AR), Estrogen Receptor (ER), Thyroid Hormone receptor, Nerve growth factor like receptors, Farnesoid X receptor (FXR), Glucocorticoid receptor (GR) progesterone receptor (PR) mineralcorticoid receptor (MR) and Retinoic Acid Receptor (RAR) (Ahmad N & Kumar R. Cancer let, 300(1), 1-9, (2011)). In response to stimulation by such a molecule the receptors can directly form a component of a biomolecular condensate and/or directly bind to DNA to regulate the expression of target genes (Sever & Glass, Cold Spring Harbor perspectives in biology vol. 5.3 a016709. 1 Mar. 2013).

As described and embodied broadly herein the first part of the first fusion protein comprises a protein that, preferably upon receiving a stimulus, translocates from one location in a cell to another location in a cell and can there become part of a biomolecular condensate. Therefore, it is highly preferred that said first part of the first fusion protein comprises a protein of any one of the herein described signaling pathways that translocates from one location in a cell to another location in a cell. Therefore, in a further embodiment a method in accordance with the invention is provided, wherein the protein comprised in the first part of the first fusion protein is a protein selected from the group consisting of β-catenin, STAT1, STAT2, STAT3, STAT4, STAT5A, STAT5B, STAT6, KRAS, BRAF, YAP, TAZ, SMAD1, SMAD2, SMAD3, SMAD5, SMAD8/9, Androgen Receptor (AR), Estrogen Receptor (ER), Farnesoid X receptor (FXR), Glucocorticoid receptor (GR), Retinoic Acid Receptor (RAR), progesterone receptor (PR), mineralcorticoid receptor (MR). Other preferred examples include ERK5, MEK5-MEKK2, P38MAPK. In a particular preferred embodiment, the first part of the first fusion protein is β-catenin. Condensation of β-catenin relies on specific amino acids in its N-, and C-terminal intrinsically disordered regions (IDRs). These are thought to engage in weak, multivalent, interactions with transcriptional cofactors in addition to the previously described strong interactions that govern β-catenin-TCF complex formation (Graham et al., Crystal structure of a beta-catenin/Tcf complex. Cell. 2000 Dec. 8; 103(6):885-96; Knapp et al., Thermodynamics of the high-affinity interaction of TCF4 with β-catenin. J. Mol. Biol. 2001 306, 1179-1189; Poy et al. Structure of a human Tcf4-beta-catenin complex. Nat Struct Biol. 2001 December; 8(12):1053-7).

In a further embodiment a method in accordance with the invention is provided, wherein the cell is selected from the group consisting of an animal cell, a vertebrate cell, an invertebrate cell, a mammalian cell, a primate cell, a rodent cell, a human cell, a primary cell, a cell line, a cancer cell, a colorectal cell, a colorectal cancer cell, a skin cell, a melanocyte, a melanoma cell, a pancreas cell, a pancreatic cancer cell, a breast cell, a breast cancer cell, a cell obtained from a patient, a cell obtained from a tumor of a patient. The methods as broadly described and embodied herein are suitable for the observing of biomolecular condensates in any type of cell wherein such biomolecular condensates are present. It is understood that in any case the cell allows for the expression of a first fusion protein, wherein the first fusion protein comprises a first part and a second part and a second fusion protein, wherein the second fusion protein comprises a first part and a second part. Accordingly, any cell that is of interest in the herein provided method can be selected in its respective assay. For example, the method is suitable for use in animal cells, e.g. vertebrate cells, such as cells of a zebrafish, invertebrate cells, e.g. Drosophila cells, or mammalian cells, such as cells of a mice, rat, primate, pig, dog, etc.

Importantly, it is contemplated that the method in accordance to the invention is suitable for use in in vivo, in vitro and in situ, and also ex vivo studies, preferably in a cell, or multiple cells, wherein biomolecular condensates are expected to be present. As broadly exemplified herein, biomolecular condensates, such as β-catenin condensates, can be observed live in the cell (e.g. in vivo) by using the method of the invention.

Further, the method of the invention allows for implementation of the method in human or humanized cell lines. This includes cells that are derived from a subject or a patient, such as of a patient suffering from a tumor. The cells may be derived from the primary tumor of said patient or from a secondary metastasized tumor. Cells may be derived from patient-derived xenografts. In some embodiments the cells are cells that have a dysregulated signaling pathway, for example are cells in which a signaling pathway is no longer regulated but has become constitutive or hyper-activated, for example as is seen in various cancers (often due to mutations in proteins, for example kinases, that form part of such signaling pathways, causing these proteins to become less or unresponsive to innate regulators of the activity of these enzymes).

In one aspect the cell is a colorectal cell, preferably a colorectal cancer cell. It is understood that canonical Wnt signaling (β-catenin dependent Wnt signaling) is upregulated in colon cancers. This is supported by the elevated levels of Wnt target genes, axin2 and human naked cuticle (hNKD) that have been determined in colorectal cancer (J. Deitrick & W. M. Pruitt, Progress in Molecular Biology and Translational Science, Academic Press, Volume 144, Pages 49-68 (2016)).

In other aspects of the current invention the cell is a skin cell, a melanocyte, a melanoma cell, a pancreas cell, a pancreatic cancer cell, a breast cell, or a breast cancer cell. Oncogenic alterations in the WNT pathway have also been observed in melanoma, pancreatic and breast cancer (Zhan T et al., Oncogene, 36(11), 1461-1473 (2017)). Therefore, in one preferred embodiment the cell wherein a first and a second fusion protein are being expressed in accordance with the method of the invention is a colorectal cancer cell, melanoma cell, breast cancer cell, pancreatic cancer cell, or any other cell in which WNT pathway alterations are part of the pathogenesis.

In a further embodiment, the invention is performed on patient derived cells, e.g. patient derived organoids. In some embodiments, the invention includes comparing the results obtained with the method of the invention between two subjects, for example a subject suffering or expected of suffering from a disease or condition, and a healthy subject or control. In some embodiments the invention includes determining the effect of a compound on condensate formation and/or dynamics that can be determined using the method of the invention, and wherein the invention is performed using patient derived material, for example a subject suffering or expected of suffering from a disease or condition.

Oncogenic pathway alterations in the TGF-β/SMAD signaling pathway have been observed in colorectal cancer and pancreatic cancer and predominantly target TGF-β receptor and SMAD4 (David CJ & Massagué J, Nat Rev Mol Cell Biol, 19(7), 419-435 (2018)). Therefore, in one preferred embodiment the cell wherein a first and a second fusion protein are being expressed in accordance with the method of the invention is a colorectal cancer cell, pancreatic cancer cell or any other cell in which TGF-β pathway alterations are part of the pathogenesis.

High JAK/STAT levels are associated with melanoma, prostate cancer, breast cancer, leukemia and lymphoma (Thomas S J et al., Br J Cancer, 113(3), 365-371 (2015)), as well as inflammatory bowel disease and severe combined immunodeficiency, multiple sclerosis psoriasis and rheumatoid arthritis (Salas A et al., Nat Rev Gastroenterol Hepatol, 17(6), 323-337, (2020), Vilklarino A V et al., Nat Immunol, 18, 374-384 (2017)). Therefore, in one preferred embodiment the cell wherein a first and a second fusion protein are being expressed in accordance with the method of the invention is a melanoma cell, prostate cancer cell, breast cancer cell, leukemia cell, lymphoma cell, immune cell or any other cell in which JAK/STAT pathway alterations are part of the pathogenesis.

Altered signaling through steroid receptors estrogen receptor, androgen receptor, progesterone receptor, glucocorticoid receptor and mineralocorticoid receptor are oncogenic driver events in breast, ovarian, lung and prostate cancer, leukemia and lymphoma (Kowalczyk W et al., Cancers, 13(9), (2021); Ahmad N & Kumar R, Cancer lett, 300(1), 1-9, (2011)). These alterations often involve mutations of the unstructured N-terminal domain that is likely involved in condensate formation (Nair S J et al., Nat Struct Mol Biol, 26(3), 193-203, (2019); Frank F, Proc Natl Acad Sci USA, 118(30) (2021)). Therefore, in one preferred embodiment the cell wherein a first and a second fusion protein are being expressed in accordance with the method of the invention is a breast cancer cell, ovarian cancer cell, lung cancer cell, prostate cancer cell, leukemia cell, lymphoma cell or any other cell in which steroid receptor pathway alterations are part of the pathogenesis.

Alterations in the RAS signaling pathway members HRAS, NRAS and KRAS occur in around 19% of patients with cancer. These cancers include the majority of identified cancers and for many of these, restoration or inhibition of RAS signaling has been shown to be beneficial (Chen K et al., J Ematol Oncol, 14(1), 116 (2021). Therefore, in one preferred embodiment the cell wherein a first and a second fusion protein are being expressed in accordance with the method of the invention is a cancer cell in which altered RAS pathway signaling is part of the pathogenesis.

Dysregulation of the Hippo pathway is associated with the hallmarks of oncogenesis and has been linked to misregulation of T-cell functionality (Ni X et al., Cancer Discov, 8, 1026-1043 (2018)). It has been shown to be altered in head and neck cancer, gynecologic cancers, gastrointestinal cancers, mesotheliomas, melanomas and a number of brain cancers (Calses P C et al., trends Cancer, 5(5), 297-307 (2019)). Therefore, in one preferred embodiment the cell wherein a first and a second fusion protein are being expressed in accordance with the method of the invention is a T-cell, head and neck cancer cell, colon cancer cell, stomach cancer cell, esophageal cancer cell, mesothelioma cell, melanoma cell, glioblastoma cell or any other cell in which dysregulation of the Hippo pathway is part of the pathogenesis.

Based on the disclosure herein, the skilled person is able to select a cell or cell line that is suitable for use in the method in accordance with the invention and that is particular suitable for the intended assay.

In a further embodiment a method in accordance with the invention is provided, wherein the cell is a cultured cell, wherein the cell culture comprises one type of cells or wherein the cell culture comprises more than one type of cells, and/or wherein the cell culture is an organoid or a spheroid. The cultured cell is herein provided by using standard culturing means and methods. The skilled person is familiar with suitable culturing material and methods for culturing a cell or cell line as meant and used herein. It is contemplated that in some embodiments, the cell culture is a mono-culture. Such a cell culture, comprising one type of cells, may be highly suitable for observing the response in said cells, and in particular the response of the formation, maintenance and/or dissolution of biomolecular condensates in said cells, to at least one stimulus, potentially more stimuli. Alternatively, it is contemplated that a co-culture or a mixed culture is used, wherein two or more cell types are cultured together so that a response of at least one, potentially more, cells to at least one, potentially more, stimulus can be observed and detected. In some embodiment, the method of the present invention is performed on an organoid or a spheroid. As provided herein the cell culture may thus also comprise 3D cell cultures that preferably resemble organ-like structures or that simulate a live cell's environmental conditions, including patient-derived tumour organoids.

In a further embodiment a method in accordance with the invention is provided, wherein the protein capable of binding to a cognate binding partner that is comprised in the second part of the first fusion protein is an antibody, even more preferred a nanobody, for example a nanobody directed to the protein comprised in the second part of the second fusion protein. As such, the first fusion protein preferably comprises 1) a protein selected from the group of proteins involved in any one of the following: Wnt signaling pathway. JAK/STAT signaling pathway, RAS signaling pathway, the HIPPO signaling pathway, TGF-β pathway or the Nuclear hormone receptor family and 2) a nanobody.

As provided herein a nanobody can refer to any single-domain antibody that is suitable for binding to at least one specified target. A suitable nanobody for use in the method of the invention is, for example, an antigen binding portion, such as a binding fragment e.g. (i) a Fab fragment, a monovalent fragment consisting of the VL, VH, CL and CH domains; (ii) a F (ab′)2 fragment, a bivalent fragment comprising two Fab fragments linked by a disulfide bond at the hinge region; (iii) a Fd fragment consisting of the VH and CH1 domains; (iv) a scFv fragment consisting of the VL and VH domains of a single arm of an antibody, and may be connected with a linker; (v) a single domain fragment (dAb fragment) which consists of a VH domain, a VH-fragment, a VL domain, or a VL-fragment, (vi) an isolated complementarity determining region (CDR); and (vii) a tandem scFv fragment consisting of 2 (or more) scFv's connected, for example head-to-tail, with a linker. A skilled person is able to include a nanobody in the fusion protein as provided herein, using conventional techniques.

It is also provided herein that said nanobody is capable of binding to a cognate binding partner, wherein said cognate binding partner is a part of the second fusion protein expressed by the same cell as wherein the first fusion protein is expressed.

In a further embodiment a method in accordance with the invention is provided, wherein the cognate binding partner is comprised in the second part of the second fusion protein. As provided herein the second fusion protein comprises two parts, wherein one part comprises a localization signal, preferably a nuclear localization signal and a second part comprises a fluorescent protein. In one preferred aspect of the invention said localization signal comprises a peptide that is native to the cell, i.e. is naturally present in the cell, and that act as a signaling fragment that mediates the transport of the second fusion protein to a desired localization in the cell (i.e. in the case of a nuclear localization signal, the second fusion protein is located in the nucleus). The first part of the second fusion protein in one preferred aspect of the method according to the invention comprises any one nuclear localization signal, such as any one of those described by Lu, J et al. Cell Commun Signal 19, 60 (2021). The second part of the second fusion protein, in embodiment of the invention, comprises the cognate binding partner, in particular the cognate binding partner to the nanobody comprised in the first fusion protein.

Therefore in a preferred embodiment a method in accordance with the invention is provided, wherein the protein capable of binding to a cognate binding partner that is comprised in the second part of the first fusion protein is a nanobody, and wherein the nanobody is directed against the fluorescent protein comprised in the second part of the second fusion protein.

As such it is understood and envisioned in the scope of the invention that in preferred embodiments of the invention, a biomolecular condensate is formed in the method of the invention wherein the biomolecular condensate is associated with the first fusion protein, comprising a first part comprising protein that forms a component of the biomolecular condensate and a second part that comprises a nanobody and wherein the first fusion protein is bound to a second fusion protein, comprising a first part comprising a localization signal and a second part comprising a fluorescent protein.

In one preferred aspect the method in accordance to the invention comprises that the fluorescent protein forming the second part of the second fusion protein is selected from the group consisting of green fluorescent protein or a GFP protein, blue fluorescent protein, yellow fluorescent protein, cyan fluorescent protein, orange fluorescent protein, red fluorescent protein, or a mCherry protein. In principle any detectable fluorescent protein can be used.

In a further embodiment a method in accordance with the invention is provided, wherein the method further comprises
providing a stimulus to the cells that causes the protein that is comprised in the first part of the first fusion protein to translocate into a cell nucleus;
removing a stimulus to the cells that causes the protein that is comprised in the first part of the first fusion protein to translocate into a cell nucleus;
providing a stimulus to the cells that inhibits the protein that is comprised in the first part of the first fusion protein to translocate into a cell nucleus; or
removing a stimulus to the cells that inhibits the protein that is comprised in the first part of the first fusion protein to translocate into a cell nucleus; or combination thereof.

In a further embodiment a method in accordance with the invention is provided, wherein the method further comprises contacting the cell with a candidate agent suspected to modulate the formation, maintenance and/or dissolution of the biomolecular condensate. It will be understood by the skilled person that such candidate agent may be provided to the cell before, during, or after formation of the biomolecular condensate and/or before, during or after providing a stimulus to the cell that causes the first fusion protein to form part of the biomolecular condensate. As will be understood by the skilled person this will not only allow the identification of agents that may modulate the dissolution of a biomolecular condensate, but also allow the identification of agents that may modulate the actual formation of such biomolecular condensate, or that may modulate the structure or function (maintenance) of an already existing biomolecular condensates. As will be understood by the skilled person, with such screening assay, both agent that enhance or that inhibit may be identified.

Therefore, in a further embodiment a screening method is provided for identifying a compound that modulates the formation, maintenance and/or dissolution of a biomolecular condensate wherein the method comprises the steps of:
(a) providing at least one cell wherein the cell expresses:
(i) a first fusion protein, wherein the first fusion protein comprises a first part and a second part, wherein the first part and the second part are linked together, wherein the first part comprises a protein that forms a component of the biomolecular condensate, wherein the second part comprises a protein capable of binding to a cognate binding partner, and
(ii) a second fusion protein, wherein the second fusion protein comprises a first part and a second part, wherein the first part and the second part are linked together, wherein the first part and/or the second part comprises the cognate binding partner, wherein the first part comprises a localization signal, wherein the second part comprises a fluorescent protein; and
(b) contacting the cell with a candidate agent; and
(c) detecting a change in the formation, maintenance, and/or dissolution of the biomolecular condensate compared to a control by detecting the fluorescent protein comprised in the second part of the second fusion protein to determine the candidate agent's modulatory activity.

As used herein “candidate agent” or “agent” refer to a molecule that may be screened for, or be identified as, modulating formation, maintenance or dissolution of the target biomolecular condensate (e.g. a β-catenin condensate). Such agent may, for example, be an inhibitor or enhancer of the formation, maintenance or dissolution of the target biomolecular condensate may find use in a variety of applications, including as therapeutic agents. The screening methods will typically be assays which provide for qualitative/quantitative measurements of the formation, maintenance or dissolution of the target biomolecular condensate in the presence of a particular candidate agent (and for example, discriminate those form agent that destroy the cell as such) For example, the assay could be an assay which measures the formation, maintenance or dissolution of the target biomolecular condensate in the presence and absence of a candidate agent, or which measures formation, maintenance or dissolution of the target biomolecular condensate in the presence of a particular candidate agent and a second compound, for example with known biological function, for example known to modulate formation, maintenance or dissolution of the target biomolecular condensate or known to modulate signaling pathways that are involved in the formation of the target biomolecular condensate.

The screening method is performed in cells and suitable formats are readily developed by those of skill in the art.

(Candidate) agents may be obtained from a wide variety of sources including libraries of synthetic or natural compounds, but also based on in-silico libraries or virtual libraries. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts or purified compounds are available or may be produced. Additionally, natural or synthetically produced libraries and compounds can be prepared using conventional chemical, physical and biochemical means, and may be used to produce combinatorial libraries. Known pharmacological agents may be subjected to directed or random chemical modifications, such as acylation, alkylation, esterification, amidification, etc. to produce structural analogs or derivates. (Candidate) agents may also be small molecules, biomolecules including peptides, saccharides, fatty acids, steroids, purines, pyrimidines, derivatives, structural analogs or combinations thereof.

Using the screening methods as disclosed herein, a variety of different therapeutic agents may be identified. Such agents may inhibitors or promoters of the targeted activity, i.e. of the formation, maintenance or dissolution of the target biomolecular condensate, where inhibitors are those agents that result in at least a reduction in the formation, maintenance or dissolution of the target biomolecular condensate as compared to a control and enhancers result in at least an increase in the formation, maintenance or dissolution of the target biomolecular condensate as compared to a control. Such agents may be used in a variety of (therapeutic) applications. As used herein, the term “determining”, for example determining formation, maintenance or dissolution of the target biomolecular condensate includes measuring, analyzing, estimating, following, and the like of such activity, for example, by using conventional means and/or techniques.

With the present invention disclosing an assay that allows direct observation of the formation, maintenance or dissolution of a biomolecular condensate, there is also provided for various screening assays that allow to identify agents that may modulate such formation, maintenance or dissolution of a biomolecular condensate. These screening assay are assays, for example, for identifying an agent that modulates the formation of a biomolecular condensate (as may, for example, be witnessed by the appearance, number or size of the target biomolecular condensate), and/or that modulates the maintenance of a biomolecular condensate (as may, for example, be witnessed by changes in the size or structure of a biomolecular condensate) or dissolution of the target biomolecular condensate (as may, for example, be witnessed by changes in the number and or size of the target biomolecular condensate).

Based on the disclosure herein the skilled person will understand how to provide for suitable formats for such screening methods and for suitable conditions under which to perform such screening methods, for example, based on the details provided in the Examples herein.

As mentioned, the candidate agents that may be tested in the various screening assays disclosed herein may be any type of molecule or mixture of molecules. The candidate agents encompass numerous chemical classes, including organic molecules, preferably small organic compounds having a molecular weight of more than 50 and less than about 2,500 daltons, antibodies, DNA and RNA molecules, other biomolecules such as peptides, saccharides, fatty acids, steroids, purines, pyrimidines, (oligo) nucleotides, know pharmaceutical drugs, derivatives, structural analogs or combinations thereof. The candidate agents may be obtained from a wide variety of sources including libraries of synthetic or natural compounds. Also contemplated are libraries of natural compounds in the form of bacterial, fungal, plant and animal extracts or natural or synthetically produced libraries and compounds, for example modified through conventional chemical, physical, virtual, computational and biochemical means.

Compounds identified with the screening assays of the invention may find use in varies fields, included, but not limited to use in the treatment of health conditions that are related to the formation, maintenance, and/or dissolution of biomolecular condensates, such as cancer.

As will be understood by the skilled person, all those aspects and preferences already discussed in the context of the method of observing the biomolecular condensates are likewise applicable to the method of the present invention that relates to screening of candidate agents. For conciseness these aspects and preferences are not to be repeated herein below but are understood to be applicable to those embodiments pertaining to the screening methods as well.

Thus, for example, in some embodiments of the screening assay the protein comprised in the first part of the first fusion protein is a protein that translocates into a cell nucleus in response to a stimulus, preferably wherein the translocation is from the plasma membrane into the cell nucleus and/or wherein the translocation is from the cytosol into the cell nucleus.

In embodiments of the screening assay, the cell is selected from the group consisting of an animal cell, a vertebrate cell, an invertebrate cell, a mammalian cell, a primate cell, a rodent cell, a human cell, a primary cell, a cell line, a cancer cell, a colorectal cell, a colorectal cancer cell, a skin cell, a melanocyte, a melanoma cell, a pancreas cell, a pancreatic cancer cell, a breast cell, a breast cancer cell, a cell obtained from a patient, a cell obtained from a tumor of a patient.

In embodiments of the screening assay the cell is a cultured cell, wherein the cell culture comprises one type of cells or wherein the cell culture comprises more than one type of cells, and/or wherein the cell culture is an organoid or a spheroid.

In embodiments of the screening assay the method further comprises
providing a stimulus to the cells that causes the protein that is comprised in the first part of the first fusion protein to translocate into a cell nucleus, and in preferred embodiments, wherein, after the translocation, the first fusion protein can become part of the biomolecular condensate;
removing a stimulus to the cells that causes the protein that is comprised in the first part of the first fusion protein to translocate into a cell nucleus (wherein the stimulus is, for example, a (known) compound that causes the protein that is comprised in the first part of the protein to translocate to the cell nucleus and become part of the biomolecular condensate);
providing a stimulus to the cells that inhibits the protein that is comprised in the first part of the first fusion protein to translocate into a cell nucleus (and where it could have become a part of a biomolecular condensate); or
removing a stimulus to the cells that inhibits the protein that is comprised in the first part of the first fusion protein to translocate into a cell nucleus (wherein by removing the stimulus that inhibits the protein that is comprised in the first part of the first fusion protein to translocate into a cell nucleus, the first fusion protein may translocate into a cell nucleus and where it may become part of a biomolecular condensate).

In embodiments of the screening assay the candidate agent is contacted with the cell before, during, or after
providing a stimulus to the cells that causes the protein that is comprised in the first part of the first fusion protein to translocate into a cell nucleus, and in preferred embodiments, wherein, after the translocation, the first fusion protein can become part of the biomolecular condensate;
   removing a stimulus to the cells that causes the protein that is comprised in the first part of the first fusion protein to translocate into a cell nucleus (wherein the stimulus is, for example, a (known) compound that causes the protein that is comprised in the first part of the protein to translocate to the cell nucleus and become part of the biomolecular condensate);
   providing a stimulus to the cells that inhibits the protein that is comprised in the first part of the first fusion protein to translocate into a cell nucleus (and where it could have become a part of a biomolecular condensate); or
   removing a stimulus to the cells that inhibits the protein that is comprised in the first part of the first fusion protein to translocate into a cell nucleus (wherein by removing the stimulus that inhibits the protein that is comprised in the first part of the first fusion protein to translocate into a cell nucleus, the first fusion protein may translocate into a cell nucleus and where it may become part of a biomolecular condensate).

In embodiments of the screening assay the detecting a change in the formation, maintenance, and/or dissolution of the biomolecular condensate comprises detecting at more than one timepoint and/or detecting within a time period of 48 hours, for example within 24 hours, 12 hours, 6 hours or less. In particular maintenance and dissolution of a biomolecular condensate may be detected within even shorter time periods such as within 60 minutes, 30 minutes, 10 minutes, 5 minutes or less. The detecting may be performed one or more times within said time period. In some embodiments, the cell are treated with the stimulus that leads to the translocation of the first fusion protein and participating on the biomolecular condensate for a period of 48 hours or less, preferably 36 hours or less, 24 hours or less, 12 hours or less, or 6 hours or less, for example, 1, 2,4, or 5 hours or less. In some embodiments, the stimulus is removed for a time period of 24 hours or less, preferably 12 hours or less, 6 hours or less, such as 120 minutes, 90 minutes, 60 minutes or 30 minutes or less. In some embodiments, in particular those relating to the method of screening, the contacting of the candidate agent with the cells is for a time period equal to that of contacting the cell with the stimulus (and preferably at the same time), or equal to the time period of that of removal of the stimulus (and preferably at the same time). In other embodiments the agent is provided to the cell after the cells has been treated with the stimulus and/or after the molecular condensates comprising the first fusion protein have formed. For example, the contacting of the agent with the cell is for a period of 48 hours or less, 24 hours or less, 12 hours or less, 6 hours or less. In a preferred embodiment the contacting of the agent with the cells is for a time period of 2 hours or less, 1 hour or less, 50, 40 or 30 minutes or less. Preferably the contacting of the candidate agent with the cell is for at least 1 minute. Preferably the contacting of the candidate agent with the cell is in the presence of the stimulus or in the absence of the stimulus.

In embodiments of the screening assay the screening assay is for identifying Wnt signaling pathway modulators, JAK/STAT signaling pathway modulators, RAS signaling pathway modulators, HIPPO signaling pathway modulators, TGF-β signaling pathway modulators, or Nuclear Receptor signaling modulators.

In a preferred embodiment a method is provided for producing a pharmaceutical composition comprising a screening method in accordance with the invention and furthermore mixing the agent identified, or a derivative or homologue thereof, with a pharmaceutically acceptable carrier.

Compounds identified with the screening assays of the invention may find use in varies fields, included, but not limited to use in the treatment of health conditions that are related to biomolecular condensates, such as, for example, cancer. Therefore there is also provided for a method for producing a pharmaceutical composition comprising a screening method as disclosed herein, in particular a screening method for identifying an agent as described herein, and furthermore mixing the agent identified, or a derivative or homologue thereof, with a pharmaceutically acceptable carrier.

Thus, the identified agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. For oral preparations, the agents can be used alone or in combination with appropriate additives to make tablets, powders, granules or capsules, for example, with conventional additives. The agents can be formulated into preparations for injection by dissolving, suspending or emulsifying them in an aqueous or nonaqueous solvent. The agents can be utilized in aerosol formulations to be administered via inhalation. Furthermore, the agents can be made into suppositories by mixing with a variety of bases such as emulsifying bases or water-soluble bases.

The pharmaceutically acceptable excipients, such as vehicles, adjuvants, carriers or diluents, are available in the art. Moreover, pharmaceutically acceptable auxiliary substances, such as pH adjusting and buffering agents, tonicity adjusting agents, stabilizers, wetting agents and the like, are also available in the art.

In another preferred embodiment at least one cell is provided, wherein the cell expresses a first fusion protein, wherein the first fusion protein comprises a first part and a second part, wherein the first part and the second part are linked together, wherein the first part comprises a protein that forms a component of the biomolecular condensate, wherein the second part comprises a protein capable of binding to a cognate binding partner, and a second fusion protein, wherein the second fusion protein comprises a first part and a second part, wherein the first part and the second part are linked together, wherein the first part and/or the second part comprises the cognate binding partner, wherein the first part comprises a localization signal, wherein the second part comprises a fluorescent protein.

In particular there is provided for a cell that comprises a biomolecular condensate wherein the biomolecular condensate is associated with the first fusion protein, comprising a first part comprising protein that forms a component of the biomolecular condensate and a second part that comprises a nanobody and wherein the first fusion protein is bound to a second fusion protein, comprising a first part comprising a localization signal and a second part comprising a fluorescent protein.

In another preferred embodiment is provided for an organoid or spheroid comprising a cell in accordance with the invention.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention.

Therefore, such adaptations and modifications are intended to be within the meaning and range of equivalents of the disclosed embodiments, based on the teaching and guidance presented herein.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references. Reference to known method steps, conventional methods steps, known methods or conventional methods is not in any way an admission that any aspect, description or embodiment of the present invention is disclosed, taught or suggested in the relevant art.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

### Example 1

A common mechanism of disease is constitutively activated signaling, which activates gene expression programs that can drive aberrant proliferation, invasion or immortality. Signaling transcription factors localize to biomolecular condensates at key cell identity genes and rely on their capacity to undergo phase separation to find and activate these genes. Here, we report a new method that allows observing the formation, maintenance and/or dissolution of such biomolecular condensates and show that disruption of such condensates, by agents, can be observed using the method disclosed herein. It was shown that such disruption, e.g. of β-catenin condensate formation, disrupts oncogenic signaling.

Intracellular signaling is a key regulator during development and adult homeostasis. As a result, dysregulation of signaling is a common cause of disease, in particular in cancer where constitutive signaling can be the initiating event driving oncogenesis. Signaling pathways culminate in the nucleus where a key signaling protein is able to activate or repress target genes. Constitutively activated signaling that results in the aberrant activation of target genes is a common driver of cancer. Major research efforts have resulted in a number of anti-cancer drugs that can inhibit upstream events in these signaling pathways (RTK/hormone therapy), but when a pathway is constitutively activated at the final step of gene activation opportunities for intervention are severely limited.

Colon cancer is the prototypical signaling-driven cancer with over 92% of cancers carrying a constitutively activating mutation in the WNT-pathway. To date, no inhibitors of WNT signaling have been clinically approved, while it has been shown that restoration of normal WNT regulation is able to effectively reverse colon cancer. WNT-driven oncogenesis is particularly challenging to target because over 5% of colon cancer has activating mutations in the CTNNB1 gene that encodes the key WNT-signaling protein beta-catenin which shuttles in the nucleus and activates transcription together with DNA-bound co-factors (Cancer Genome Atlas Network, 2012). Therefore, the holy grail of WNT-inhibition has been the disruption of the protein complex formed by beta-catenin and its DNA-resident recruiting factor TCF/LEF, but these efforts have been so far unsuccessful.

Recently we and others have shown that beta-catenin, like a number of transcription factors, partitions into biomolecular condensates with transcriptional machinery and that this is necessary for efficient transcriptional activation. In general, biomolecular condensates have emerged as a major organizational mechanism in the cell. However, the formation and dissolution of condensates is still poorly understood. The major research focus has been the identification of the molecular features that lead to condensate formation but little is known about the cellular mechanisms that can inhibit it. Perturbing condensates is currently actively explored as a potential therapeutical modality and a better understanding of the cellular mechanisms that regulate condensation can inform these efforts.

### In Vivo Detection of Condensates as Exemplified by β-Catenin Condensates

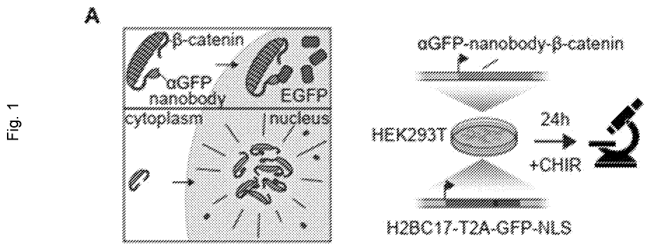

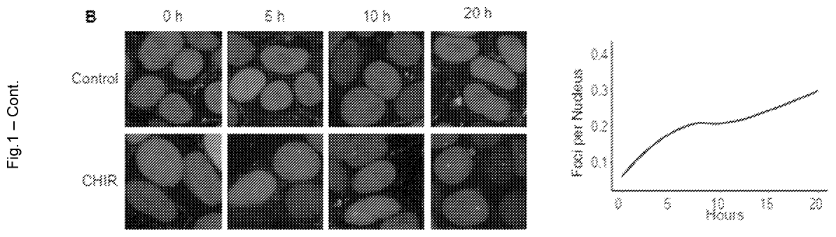

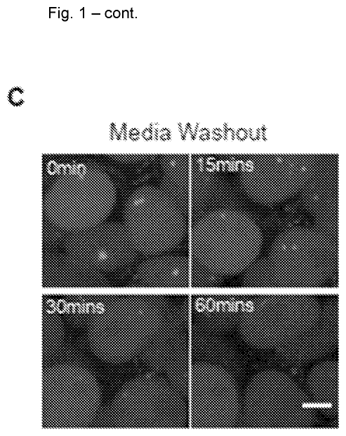

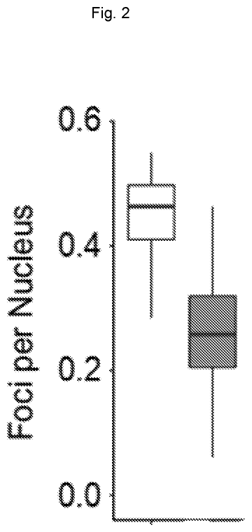

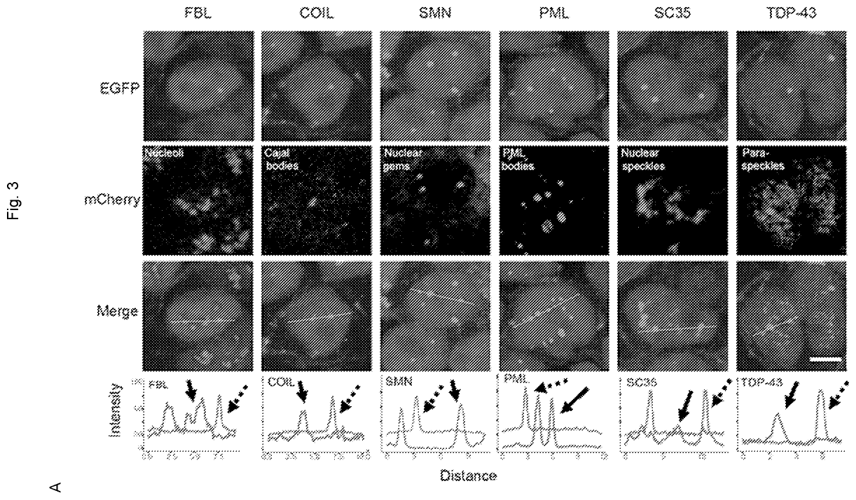

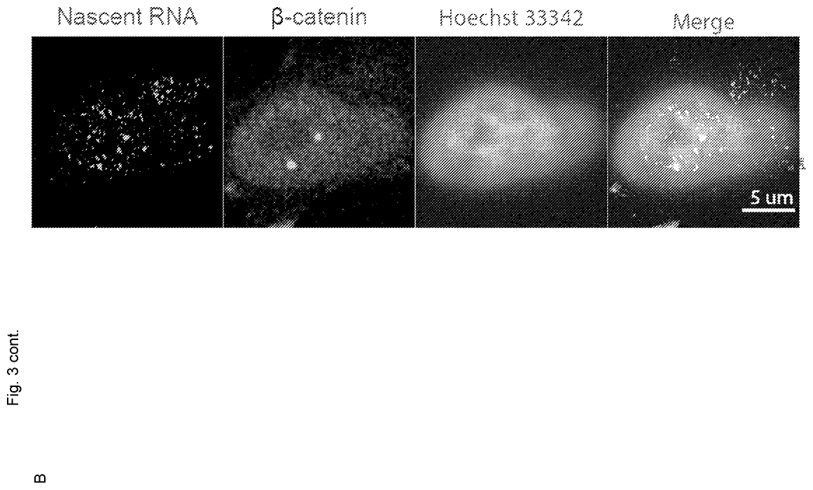

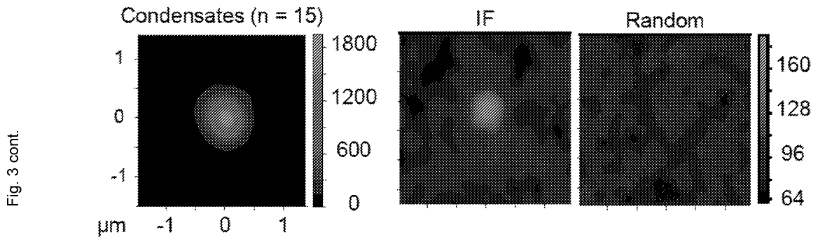

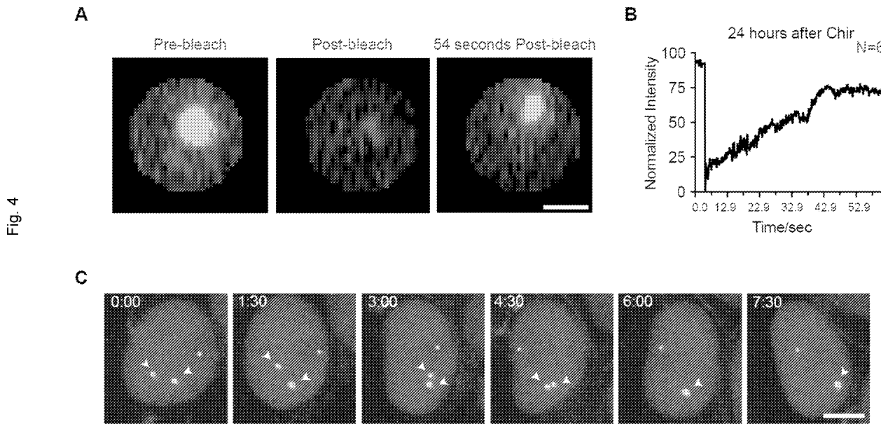

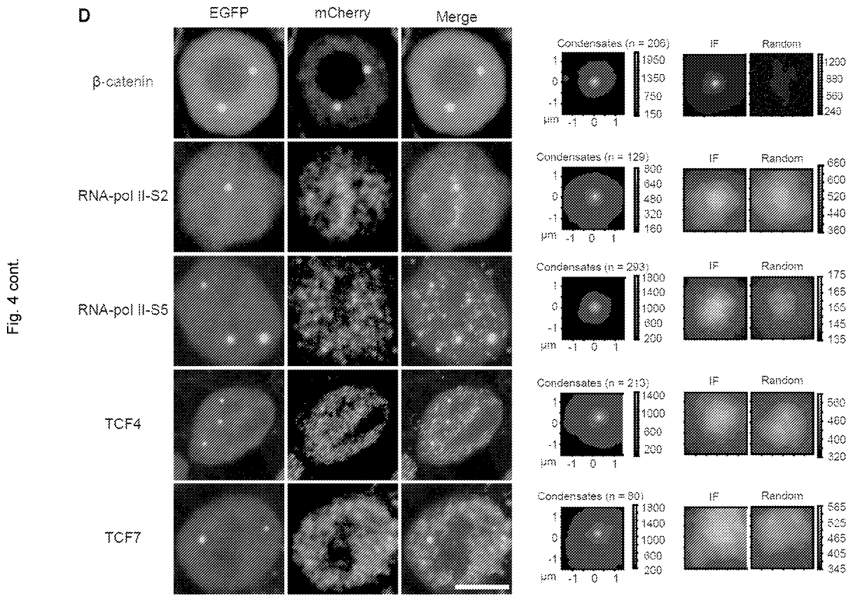

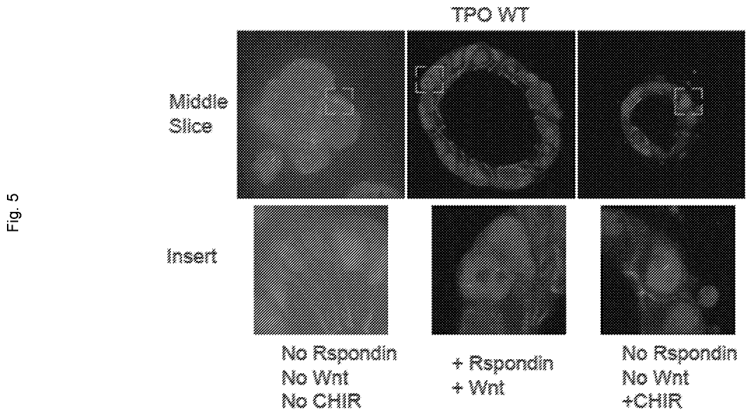

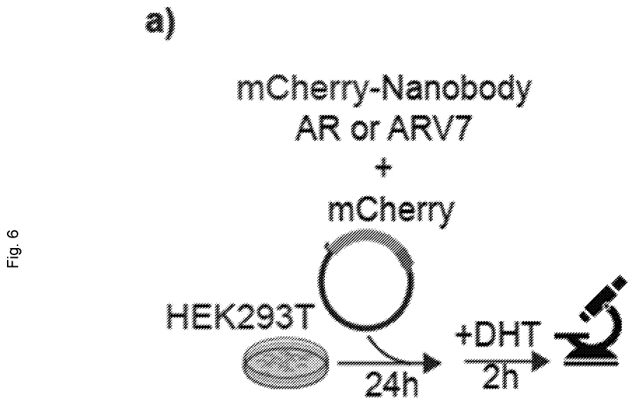

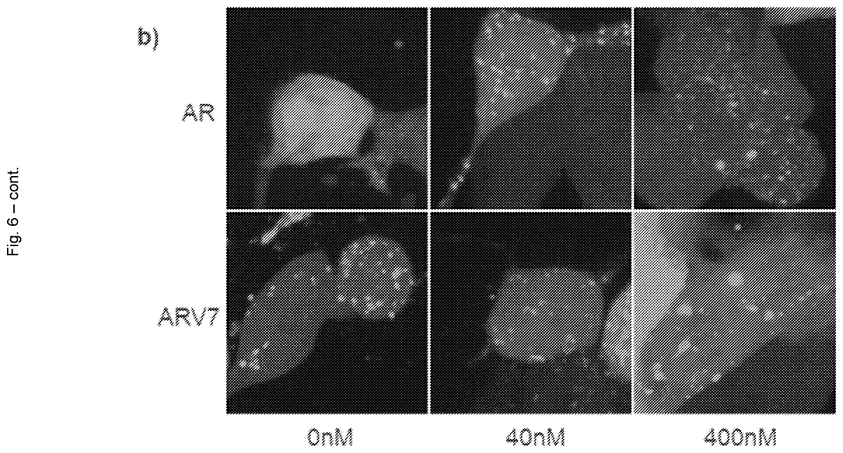

To detect β-catenin condensates in vivo we generated a decoupled, active-only β-catenin imaging system (FIG. 1A). The majority of β-catenin is present in the plasma membrane where it is involved in cell-cell adhesion, which makes nuclear visualization of β-catenin-condensates through direct labeling challenging. Furthermore, direct labeling of proteins with rapid turnover times like β-catenin might fail to detect all labeled protein because of degradation prior to complete fluorophore maturation (Dickinson D J et al. Dev Cell 32 416-434 (2017), Hazelrigg T et al. Dev Biol 199 245-249 (1998), Little S C et al. PLOS Biol 9 e1000596 (2011), Wacker S A et al. Dev Dyn 236 (2007)). Therefore, EGFP was expressed coupled to a nuclear localization signal (NLS) through integration in the locus of the moderately expressed H2B gene copy H2BC17, using a T2A, for example separated by the T2A sequence. In these same cells we coupled a high affinity nanobody against EGFP (Prole D L et al., BMC biol 17(1) 41 (2019)) to β-catenin by integration into the endogenous locus (FIG. 1A). In this imaging system the EGFP is predominantly nuclear and only labels β-catenin when the WNT pathway is activated and β-catenin translocates to the nucleus as well (FIG. 1A). Upon WNT activation of GFP Nanobody β-catenin HEK293 cells we observed the appearance of several β-catenin condensates, or GNb β-catenin condensates in the nucleus of stimulated cells over a period of 20 hours (FIG. 1B). A maximum was reached after about 5 hours. These condensates are spherical, exhibit fast recovery kinetics measured by fluorescence recovery after photobleaching (FRAP) (FIG. 4A, B) and fuse upon contact (FIG. 4C). Importantly, when WNT stimulation was withdrawn these condensates dissolved within approximately 30 minutes (FIG. 1C). Co-staining of well-established nuclear compartments showed that the β-catenin condensates did not co-localize with Nucleoli, Cajal bodies, Nuclear gems, PML bodies Nuclear speckles or Para-speckles, but form their own distinct compartments (FIG. 3A), for example, that co-localize with TCF/LEF family members endogenous to these cells. These results show that the GNB system (GFP Nanobody system) can rapidly detect the WNT-responsive formation and dissolution of biomolecular condensates such as β-catenin condensates in live cells.

Importantly, when WNT stimulation was withdrawn these condensates dissolved within approximately 30 minutes (FIG. 1C). These results show that the can rapidly detect the WNT-responsive formation and dissolution of β-catenin condensates in live cells.

To test if the detected b-catenin condensates transcriptionally active we co-stained for active RNA Polymerase II (poIII) and for the DNA-resident b-catenin recruiting factors TCF7 and TCF7L2. As has been observed before, active poIII is present in distinct condensates throughout the nucleus and all b-catenin condensates overlap with active poIII condensates, and one of the antibodies showed a specific enrichment over random control (FIG. 4D). Both TCF7 and TCF7L2 staining's also showed a high number of nuclear condensates and all b-catenin condensates overlap with at least one TCF condensate (FIG. 4D). Because of the high number of TCF7 and TCF7L2 condensates there was no enrichment observed at b-catenin condensates. Taken together, these data show that b-catenin condensates co-localize with both TCF family member condensates and active poIII condensates, indicating the b-catenin condensates are transcriptionally active. To test if b-catenin condensates are sites of RNA production we labeled nascent RNA with a pulse of 5-ethynyl uridine (EU) that was subsequently coupled to a fluorescent dye. A 10 minute-pulse lead to the detection of several nascent RNA clusters in the nucleus of stimulated GNB cells, with several clusters of distinctly larger size than the average. These large clusters invariably overlapped with b-catenin condensates (FIG. 3B,C). These data show that b-catenin condensates are sites of active transcription.

Taken together, these results show that b-catenin condensates detected with the GNB system of the invention are distinct form other common condensates, behave like liquid droplets and are associated with active transcription. These characteristics make them exceptionally suited as a platform for detection of condensate perturbating compounds.

FIG. 2 shows results obtained with an agent that was identified to promote dissolution of β-catenin condensates, as witnessed by the reduced number of foci per nucleus in the presence of the compound (right) in comparison to control (left). Treatment of cells with said component lead to dissolution as can be observed using the method described herein. Live-cell time-course imaging revealed that the compound was able to reduce the life time of persisting β-catenin condensates by approximately half and reduce the size of newly forming condensates by more than 70%. To test if the compound had an effect on β-condensates in cancer, the compound was provided to colorectal cancer cells with endogenously labeled β-catenin, that contain a monoallelic oncogenic S45 mutation (a constitutive, oncogenic activation of the WNT-pathway. This is a mutation that prevents normal b-cat degradation, which leads to accumulation and translocation of b-cat). Also, in these cancer cells, the compound, but not control, reduced the intensity of β-catenin condensates when analyzing the foci that were able to be called individually or analyzing the total nuclear intensity. These results indicate that the compound disrupts β-catenin condensates in physiological and oncogenic conditions. It was also shown that perturbation of β-catenin condensates by the compound inhibits oncogenic WNT-driven expression in colon cancer cells.

These results show that with the method of the invention candidate agents can be identified that modulate formation, maintenance and/or dissolution of biomolecular condensates, as exemplified by the dissolution of the β-catenin condensates in the presence of the compound.

FIG. 5 also shows the invention, exemplified by the GFP-nanobody-βcatenin system can be used on organoids, including organoids using patient material.

### Experimental Methods and Models

### Cell Lines

HCT 116 Human Colorectal Cancer cells (Male origin) and 293T Human Embryonic Kidney cells (HEK293T, Fetal origin) were obtained from the ATCC and cultured at 37° C. and 5% CO2 in high glucose (4.5 g/L) DMEM supplemented with 100U/ml penicillin (Lonza), 100 mg/ml streptomycin (Lonza) and 10% FBS (Lonza). For CHIR treatment, cells were seeded for the appropriate experiment and maintained in fresh complete media with 3 μM CHIR for 24h.

### HEK293T H2BC17-T2A-EGFP GNB-β-Catenin Cell Lines Generation

HEK293T embryonic kidney cells were genetically modified using the CRISPR-Cas9 system. A guide targeting the C terminus of H2BC 17 (Human H2B clustered histone 17) was cloned into a px330 vector with an mCherry selectable marker and the following sequence: GGCAGCTGCGAGAGCTCACT (SEQ ID NO: 1). A repair template with 800-1000 bp homology to the endogenous locus flanking T2A (2A peptide from Thosea asigna virus capsid protein), EGFP and SV40 NLS (nuclear localization signal) was cloned into a pUC19 vector. Cells were transfected with both constructs and sorted for mCherry two days post-transfection to single cells in 96-well plates. Positive H2BC17-T2A-EGFP clonal cell lines were selected by fluorescence microscope for EGFP expression and validated by genomic PCR. A guide targeting the N-terminus of β-catenin was cloned into a px330 vector with an mCherry selectable marker and the following sequence: CTGCGTGGACAATGGCTACT (SEQ ID NO:2).

A repair template with 800 bp homology to the endogenous locus flanking an GFP-nanobody (GNB) was cloned into a pUC19 vector. GNB (GFP-nanobody) fragment was PCR from ssGNb-mCherry construct (Addgene, 128788). H2BC17-T2A-EGFP Cells were transfected with both constructs and sorted for mCherry two days post-transfection to single cells in 96-well plates. Genomic PCR was conducted to select positive H2BC17-T2A-EGFP GNB-β-catenin clonal cell lines.

### FRAP

For nuclear β-catenin foci, FRAP was performed on Zeiss LSM880 microscope with an alpha Plan-Fluar 100×/1.49 Oil M27 objective by 488 nm laser. Bleaching was performed using 100% laser power and images were collected every 100 ms for 1 min. Fluorescence intensity was measured using Fiji. Background intensity was subtracted, and values were reported relative to pre-bleaching time points.

### Immunofluorescence (IF)

Cells were seeded at 70-80% confluence in a 15 mm glass cover slide, pre-coated for 5 mins with 10 μg/mL poly-L-Lysine. After cells attachment, appropriate treatments were added to the medium for 24h. Subsequently, cells were washed twice with PBS and fixed for 15 mins at room temperature with 4% paraformaldehyde (PFA) in PBS, washed 2 times with PBS and permeabilized with 0.1% Triton-X100 in PBS for 5 min room temperature. Cover slides were washed twice again with PBS and blocked for 30 mins with purified 1:10000 IgG in 2% BSA in PBS. Cells were then incubated with appropriated primary antibodies (see figures) for 1h room temperature or overnight at 4° C., washed 6 times with PBS and incubated with secondary antibodies for 1h room temperature, washed 6 more times with PBS and then mounted using the slide mounting media Epredia Immu-Mount (Fisher). Images were acquired by scanning a series of Z stacks on the Nikon spinning disk confocal system and analyzed by the ImageJ Software.

### Live Cell Microscopy

Live cells experiments were performed on a Nikon spinning disk confocal attached to cell culture system in 5% CO2 and 37° C. humidified chamber. The focal plane was monitored and addressed by the Z drift compensator system. Images were acquired using an Apo TIRF 60× Oil DIC N2 lens with Z series and time-series. Time Lapses were performed with 15 min interval for 96 cycles. Raw images were processed and analyzed using ImageJ software.

### Quantification and Statistical Analysis

All experiments were performed with technical and biological duplicates or triplicates. Biological replicate sample sizes (n=x) and other experiment-specific details are indicated in the figure legends. Data were plotted and analyzed using GraphPad or R software. Data are represented as the mean of multiple replicates±SD or SEM.

### Example 2

This example shows the invention using a mCherry-nanobody system for the Androgen Receptor pathway. A schematic representation of the experiment is shown in FIG. 6. HEK293T cells were co-transfected with mCherry together with either mCherry-nanobody-AR or the splicing variant ARV7, which is constitutively active. After one day, cells were treated with different concentrations of Dihydrotestosterone (DHT). b) Representative images of mCherry condensates seen in the cells. This experiment shows the invention is broadly applicable to different types of condensates, including disease relevant condensate. The experiment also shows the invention is broadly applicable using different types of fluorescent proteins or fluorophores.

### Material and Methods

### Organoids Culture:

Under normal conditions, organoids were grown in Cultrex™ Basement Membrane Extract (BME) domes supplemented with organoid media: DMEM/F12 media supplemented with 20 mM Glutamax, 10 mM Hepes and Penicillin/Streptomycin, 0.5 nM Wnt Surrogate, 20% Rspondin conditioned media, 10% Noggin conditioned media, 50 ng/ml EGF, 1×B27 supplement, 1.25 mM N-acetyl-L-Cystein, 10 mM Nicotinamide, 500 nM A83-01 and 10 uM SB202190. 3 days before imaging, the WNT-surrogate and R-spondin were removed from the media and then added, when indicated, 24 hours before imaging. 3 uM of CHIR was added, when indicated, 24 hours before imaging. Images were taken using a Zeiss LSM880 confocal microscope with an alpha Plan-Fluar 63×/1.49 Oil M27 objective or a Nikon spinning disk using an Apo TIRF 60× Oil DIC N2 lens.

### Androgen Receptor Condensates

A mCherry-nanobody was designed to bind to the N-terminus of mCherry, the sequence can be found bellow. The nanobody was then, cloned to a construct containing the Androgen receptor (AR) and its splicing variant ARV7—common found in resistant prostate cancer-tagged to sfGFP. HEK293T cells were co-transfected transiently with AR/ARV7-sfGFP-mCherry-nanobody together with mCherry using the TransIT transfection reagent, according to the manufacturer instructions. Cells were then, serum starved for 48h prior dihydrotestosterone (DHT) treatment and imaged, after 2 hours of treatment, on a Nikon spinning disk using an Apo TIRF 60× Oil DICN2 lens.

### Constructs:

mCherry-nanobody (SEQ ID NO: 3):
atggctcaggtgcagctggtggaaagcggcggctctctggtgcagcctggcggatctctgagactgagctgtgccgcc
agcggcagatttgccgagagcagcagcatgggctggttcagacaggcccctggcaaagaacgcgagttcgtggcc
gccatctcttggagcggcggagccaccaattacgccgatagcgccaagggccggttcaccctgagccgggacaac
accaagaacaccgtgtacctgcagatgaacagcctgaagcccgacgacaccgccgtgtactactgcgccgccaac
ctgggcaactacatcagcagcaaccagcggctgtacggctactggggccagggaacacaagtgaccgtgtccagc
cctttcacaggcggcggaggatcc
sfGFP (SEQ ID NO: 4).:
agcaaaggagaagaacttttcactggagttgtcccaattcttgttgaattagatggtgatgttaatgggcacaaattttctg
tccgtggagagggtgaaggtgatgctacaaacggaaaactcacccttaaatttatttgcactactggaaaactacctgtt
ccgtggccaacacttgtcactactctgacctatggtgttcaatgcttttcccgttatccggatcacatgaaacggcatgact
ttttcaagagtgccatgcccgaaggttatgtacaggaacgcactatatctttcaaagatgacgggacctacaagacgc
gtgctgaagtcaagtttgaaggtgatacccttgttaatcgtatcgagttaaagggtattgattttaaagaagatggaaaca
ttcttggacacaaactcgagtacaactttaactcacacaatgtatacatcacggcagacaaacaaaagaatggaatc
aaagctaacttcaaaattcgccacaacgttgaagatggttccgttcaactagcagaccattatcaacaaaatactcca
attggcgatggccctgtccttttaccagacaaccattacctgtcgacacaatctgtcctttcgaaagatcccaacgaaaa
gcgtgaccacatggtccttcttgagtttgtaactgctgctgggattacacatggcatggatgagctctacaaa
AR (SEQ ID NO: 5).:
gaagtgcagttagggctgggaagggtctaccctcggccgccgtccaagacctaccgaggagctttccagaatctgttc
cagagcgtgcgcgaagtgatccagaacccgggccccaggcacccagaggccgcgagcgcagcacctcccggcg
ccagtttgctgctgctgcagcagcagcagcagcagcagcagcagcagcagcagcagcagcagcagcagcagca
gcagcagcagcaagagactagccccaggcagcagcagcagcagcagggtgaggatggttctccccaagcccatc
gtagaggccccacaggctacctggtcctggatgaggaacagcaaccttcacagccgcagtcggccctggagtgcca
ccccgagagaggttgcgtcccagagcctggagccgccgtggccgccagcaaggggctgccgcagcagctgccag
cacctccggacgaggatgactcagctgccccatccacgttgtccctgctgggccccactttccccggcttaagcagctg
ctccgctgaccttaaagacatcctgagcgaggccagcaccatgcaactccttcagcaacagcagcaggaagcagta
tccgaaggcagcagcagcgggagagcgagggaggcctcgggggctcccacttcctccaaggacaattacttaggg
ggcacttcgaccatttctgacaacgccaaggagttgtgtaaggcagtgtcggtgtccatgggcctgggtgtggaggcgt
tggagcatctgagtccaggggaacagcttcggggggattgcatgtacgccccacttttgggagttccacccgctgtgcg
tcccactccttgtgccccattggccgaatgcaaaggttctctgctagacgacagcgcaggcaagagcactgaagata
ctgctgagtattcccctttcaagggaggttacaccaaagggctagaaggcgagagcctaggctgctctggcagcgctg
cagcagggagctccgggacacttgaactgccgtctaccctgtctctctacaagtccggagcactggacgaggcagct
gcgtaccagagtcgcgactactacaactttccactggctctggccggaccgccgccccctccgccgcctccccatccc
cacgctcgcatcaagctggagaacccgctggactacggcagcgcctgggcggctgcggcggcgcagtgccgctat
ggggacctggcgagcctgcatggcgcgggtgcagcgggacccggttctgggtcaccctcagccgccgcttcctcatc
ctggcacactctcttcacagccgaagaaggccagttgtatggaccgtgtggtggtggtgggggtggtggcggcggcg
gcggcggcggcggcggcggcggcggcggcggcggcggcggcgaggcgggagctgtagccccctacggctaca
ctcggccccctcaggggctggcgggccaggaaagcgacttcaccgcacctgatgtgtggtaccctggcggcatggtg
agcagagtgccctatcccagtcccacttgtgtcaaaagcgaaatgggcccctggatggatagctactccggaccttac
ggggacatgcgtttggagactgccagggaccatgttttgcccattgactattactttccaccccagaagacctgcctgat
ctgtggagatgaagcttctgggtgtcactatggagctctcacatgtggaagctgcaaggtcttcttcaaaagagccgctg
aagggaaacagaagtacctgtgcgccagcagaaatgattgcactattgataaattccgaaggaaaaattgtccatctt
gtcgtcttcggaaatgttatgaagcagggatgactctgggagcccggaagctgaagaaacttggtaatctgaaactac
aggaggaaggagaggcttccagcaccaccagccccactgaggagacaacccagaagctgacagtgtcacacatt
gaaggctatgaatgtcagcccatctttctgaatgtcctggaagccattgagccaggtgtagtgtgtgctggacacgaca
acaaccagcccgactcctttgcagccttgctctctagcctcaatgaactgggagagagacagcttgtacacgtggtca
agtgggccaaggccttgcctggcttccgcaacttacacgtggacgaccagatggctgtcattcagtactcctggatggg
gctcatggtgtttgccatgggctggcgatccttcaccaatgtcaactccaggatgctctacttcgcccctgatctggttttca
atgagtaccgcatgcacaagtcccggatgtacagccagtgtgtccgaatgaggcacctctctcaagagtttggatggc
tccaaatcaccccccaggaattcctgtgcatgaaagcactgctactcttcagcattattccagtggatgggctgaaaaat
caaaaattctttgatgaacttcgaatgaactacatcaaggaactcgatcgtatcattgcatgcaaaagaaaaaatccca
catcctgctcaagacgcttctaccagctcaccaagctcctggactccgtgcagcctattgcgagagagctgcatcagtt
cacttttgacctgctaatcaagtcacacatggtgagcgtggactttccggaaatgatggcagagatcatctctgtgcaag
tgcccaagatcctttctgggaaagtcaagcccatctatttccacacccagtga
ARV7 (SEQ ID NO: 6).:
atggaagtgcagttagggctgggaagggtctaccctcggccgccgtccaagacctaccgaggagctttccagaatct
gttccagagcgtgcgcgaagtgatccagaacccgggccccaggcacccagaggccgcgagcgcagcacctcccg
gcgccagtttgctgctgctgcagcagcagcagcagcagcagcagcagcagcagcagcagcagcagcagcagca
gcagcagcagcagcaagagactagccccaggcagcagcagcagcagcagggtgaggatggttctccccaagcc
catcgtagaggccccacaggctacctggtcctggatgaggaacagcaaccttcacagccgcagtcggccctggagt
gccaccccgagagaggttgcgtcccagagcctggagccgccgtggccgccagcaaggggctgccgcagcagctg
ccagcacctccggacgaggatgactcagctgccccatccacgttgtccctgctgggccccactttccccggcttaagc
agctgctccgctgaccttaaagacatcctgagcgaggccagcaccatgcaactccttcagcaacagcagcaggaag
cagtatccgaaggcagcagcagcgggagagcgagggaggcctcgggggctcccacttcctccaaggacaattact
tagggggcacttcgaccatttctgacaacgccaaggagttgtgtaaggcagtgtcggtgtccatgggcctgggtgtgga
ggcgttggagcatctgagtccaggggaacagcttcggggggattgcatgtacgccccacttttgggagttccacccgct
gtgcgtcccactccttgtgccccattggccgaatgcaaaggttctctgctagacgacagcgcaggcaagagcactga
agatactgctgagtattcccctttcaagggaggttacaccaaagggctagaaggcgagagcctaggctgctctggca
gcgctgcagcagggagctccgggacacttgaactgccgtctaccctgtctctctacaagtccggagcactggacgag
gcagctgcgtaccagagtcgcgactactacaactttccactggctctggccggaccgccgccccctccgccgcctccc
catccccacgctcgcatcaagctggagaacccgctggactacggcagcgcctgggcggctgcggcggcgcagtgc
cgctatggggacctggcgagcctgcatggcgcgggtgcagcgggacccggttctgggtcaccctcagccgccgcttc
ctcatcctggcacactctcttcacagccgaagaaggccagttgtatggaccgtgtggtggtggtgggggtggtggcgg
cggcggcggcggcggcggcggcggcggcggcggcggcggcggcggcgaggcgggagctgtagccccctacgg
ctacactcggccccctcaggggctggcgggccaggaaagcgacttcaccgcacctgatgtgtggtaccctggcggc
atggtgagcagagtgccctatcccagtcccacttgtgtcaaaagcgaaatgggcccctggatggatagctactccgga
ccttacggggacatgcgtttggagactgccagggaccatgttttgcccattgactattactttccaccccagaagacctg
cctgatctgtggagatgaagcttctgggtgtcactatggagctctcacatgtggaagctgcaaggtcttcttcaaaagag
ccgctgaagggaaacagaagtacctgtgcgccagcagaaatgattgcactattgataaattccgaaggaaaaattgt
ccatcttgtcgtcttcggaaatgttatgaagcagggatgactctgggagaaaaattccgggttggcaattgcaagcatct
caaaatgaccagaccctga

1. A method of observing biomolecular condensates, preferably nuclear biomolecular condensates, wherein the method comprises:
(a) Providing at least one cell wherein the cell expresses
(i) a first fusion protein, wherein the first fusion protein comprises a first part and a second part,
wherein the first part and the second part are linked together,
wherein the first part comprises a protein that forms a component of the biomolecular condensate,
wherein the second part comprises a protein capable of binding to a cognate binding partner,
and
(ii) a second fusion protein, wherein the second fusion protein comprises a first part and a second part,
wherein the first part and the second part are linked together,
wherein the first part and/or the second part comprises the cognate binding partner,
wherein the first part comprises a localization signal,
wherein the second part comprises a fluorescent protein; and
(b) observing the presence of the biomolecular condensates by detecting the fluorescent protein comprised in the second part of the second fusion protein.

2. The method according to claim 1 wherein the protein comprised in the first part of the first fusion protein is a protein that translocates from a first cellular compartment into a second cellular compartment, preferably into a cell nucleus in response to a stimulus, preferably wherein the translocation is from the plasma membrane into the cell nucleus and/or wherein the translocation is from the cytosol into the cell nucleus.

3. The method according to claim 1, wherein the protein comprised in the first part of the first fusion protein is a protein selected from the group consisting of a protein of the Wnt signaling pathway, JAK/STAT signaling pathway, RAS signaling pathway, HIPPO signaling pathway TGF-β pathway or the Nuclear hormone receptor family.

4. The method according to claim 1, wherein the protein comprised in the first part of the first fusion protein is a protein selected from the group consisting of β-catenin, STAT1, STAT2, STAT3, STAT4, STAT5A, STAT5B, STAT6, KRAS, BRAF, YAP, TAZ, SMAD1, SMAD2, SMAD3, SMAD5, SMAD8/9, Androgen Receptor (AR), Estrogen Receptor (ER), Farnesoid X receptor (FXR), Glucocorticoid receptor (GR), Retinoic Acid Receptor (RAR), or ortholog thereof.

5. The method according to claim 1, wherein the cell is selected from the group consisting of an animal cell, a vertebrate cell, an invertebrate cell, a mammalian cell, a primate cell, a rodent cell, a human cell, a primary cell, a cell line, a cancer cell, a colorectal cell, a colorectal cancer cell, a skin cell, a melanocyte, a melanoma cell, a pancreas cell, a pancreatic cancer cell, a breast cell, a breast cancer cell, a cell obtained from a patient, a cell obtained from a tumor of a patient.

6. The method according to claim 1, wherein the cell is a cultured cell, wherein the cell culture comprises one type of cells or wherein the cell culture comprises more than one type of cells, and/or wherein the cell culture is a 3D cell culture system, preferably organoid and/or a spheroid, including a patient derived organoid.

7. The method according to claim 1, wherein the stimulus that causes the protein that is comprised in the first part of the first fusion protein to translocate into a cell nucleus is selected from the group consisting of a wnt signaling agonist, a cytokine JAK/STAT agonist, a EGF pathway agonist, a TGF-b signaling agonist, a hippo signaling agonist, a nuclear hormone agonist.

8. The method according to claim 1, wherein the protein capable of binding to a cognate binding partner that is comprised in the second part of the first fusion protein is a nanobody.

9. The method according to claim 1, wherein the cognate binding partner is comprised in the second part of the second fusion protein.

10. The method according to claim 1, wherein the protein capable of binding to a cognate binding partner that is comprised in the second part of the first fusion protein is a nanobody, and wherein the nanobody is directed against the fluorescent protein comprised in the second part of the second fusion protein, preferably wherein the fluorescent protein is selected from the group consisting of green fluorescent protein, blue fluorescent protein, yellow fluorescent protein, cyan fluorescent protein, orange fluorescent protein, red fluorescent protein, or a mCherry protein.

11. The method according to claim 1, wherein the method further comprises
providing a stimulus to the cells that causes the protein that is comprised in the first part of the first fusion protein to translocate into a cell nucleus;
removing a stimulus to the cells that causes the protein that is comprised in the first part of the first fusion protein to translocate into a cell nucleus;
providing a stimulus to the cells that inhibits the protein that is comprised in the first part of the first fusion protein to translocate into a cell nucleus; or
removing a stimulus to the cells that inhibits the protein that is comprised in the first part of the first fusion protein to translocate into a cell nucleus.

12. The method according to claim 11, wherein the method further comprises contacting the cell with a candidate agent suspected to modulate the formation, maintenance and/or dissolution of the biomolecular condensate.

13. A screening method for identifying a compound that modulates the formation, maintenance and/or dissolution of a biomolecular condensate, wherein the method comprises
(a) providing at least one cell wherein the cell expresses
(i) a first fusion protein, wherein the first fusion protein comprises a first part and a second part,
wherein the first part and the second part are linked together,
wherein the first part comprises a protein that forms a component of the biomolecular condensate,
wherein the second part comprises a protein capable of binding to a cognate binding partner,
and
(ii) a second fusion protein, wherein the second fusion protein comprises a first part and a second part,
wherein the first part and the second part are linked together,
wherein the first part and/or the second part comprises the cognate binding partner,
wherein the first part comprises a localization signal,
wherein the second part comprises a fluorescent protein;
(b) contacting the cell with a candidate agent; and
(c) detecting a change in the formation, maintenance, and/or dissolution of the biomolecular condensate, compared to a control by detecting the fluorescent protein comprised in the second part of the second fusion protein to determine the candidate agent's modulatory activity.

14. The method according to claim 13, wherein the protein comprised in the first part of the first fusion protein is a protein that translocates from a first cellular compartment into a second cellular compartment, preferably into a cell nucleus in response to a stimulus, preferably wherein the translocation is from the plasma membrane into the cell nucleus and/or wherein the translocation is from the cytosol into the cell nucleus.

15. The method according to claim 13, wherein the cell is selected from the group consisting of an animal cell, a vertebrate cell, an invertebrate cell, a mammalian cell, a primate cell, a rodent cell, a human cell, a primary cell, a cell line, a cancer cell, a colorectal cell, a colorectal cancer cell, a skin cell, a melanocyte, a melanoma cell, a pancreas cell, a pancreatic cancer cell, a breast cell, a breast cancer cell, a cell obtained from a patient, a cell obtained from a tumor of a patient.

16. The method according to claim 13, wherein the cell is a cultured cell, wherein the cell culture comprises one type of cells or wherein the cell culture comprises more than one type of cells, and/or wherein the cell culture is an organoid or a spheroid.

17. The method according to claim 13, wherein the method further comprises
providing a stimulus to the cells that causes the protein that is comprised in the first part of the first fusion protein to translocate into a cell nucleus;
removing a stimulus to the cells that causes the protein that is comprised in the first part of the first fusion protein to translocate into a cell nucleus;
providing a stimulus to the cells that inhibits the protein that is comprised in the first part of the first fusion protein to translocate into a cell nucleus; or
removing a stimulus to the cells that inhibits the protein that is comprised in the first part of the first fusion protein to translocate into a cell nucleus.

18. The method according to claim 13, wherein the candidate agent is contacted with the cell before, during, or after
providing a stimulus to the cells that causes the protein that is comprised in the first part of the first fusion protein to translocate into a cell nucleus;
removing a stimulus to the cells that causes the protein that is comprised in the first part of the first fusion protein to translocate into a cell nucleus;
providing a stimulus to the cells that inhibits the protein that is comprised in the first part of the first fusion protein to translocate into a cell nucleus; or
removing a stimulus to the cells that inhibits the protein that is comprised in the first part of the first fusion protein to translocate into a cell nucleus.

19. The method according to claim 13, wherein the detecting a change in the formation, maintenance, and/or dissolution of the biomolecular condensate comprises detecting at more than one timepoint and/or detecting within a time period of 48 hours, preferably within 24 hours or less, 12 hours or less, 6 hours or less such as 60 minutes or less, 30 minutes or less, 10 minutes or less or 5 minutes or less.

20. The method according to claim 13, for identifying Wnt signaling pathway modulators, JAK/STAT signaling pathway modulators, RAS signaling pathway modulators, HIPPO signaling pathway modulators, TGF-β signaling pathway modulators, or Nuclear Receptor signaling modulators.

21. A method for producing a pharmaceutical composition comprising a screening method according to any of the previous claims and furthermore mixing the agent identified, or a derivative or homologue thereof, with a pharmaceutically acceptable carrier.

22. At least one cell wherein the cell expresses
(i) a first fusion protein, wherein the first fusion protein comprises a first part and a second part, wherein the first part and the second part are linked together, wherein the first part comprises a protein that forms a component of the biomolecular condensate,
wherein the second part comprises a protein capable of binding to a cognate binding partner,
(ii) a second fusion protein, wherein the second fusion protein comprises a first part and a second part, wherein the first part and the second part are linked together, wherein the first part and/or the second part comprises the cognate binding partner,
wherein the first part comprises a localization signal, preferably a nuclear localization signal,
wherein the second part comprises a fluorescent protein.

23. An organoid and/or spheroid comprising a cell according to claim 22.
